(19) United States
(12) Patent Application Publication
(10) Pub. No.: US 2025/0170222 A1
(43) Pub. Date: May 29, 2025

(54) SUPER-OXIDE DISMUTASE SOLUBLE FIBER COMPOSITIONS AND METHODS OF USE

(71) Applicant: ADVANCE PHARMACEUTICAL, INC., Holtsville, NY (US)

(72) Inventor: Liaquat HOSSAIN, Holtsville, NY (US)

(21) Appl. No.: 18/842,692

(22) PCT Filed: Mar. 1, 2023
(86) PCT No.: PCT/US2023/063477

§ 371 Date: Aug. 29, 2024

Related U.S. Application Data
(60) Provisional application No. 63/315,631, filed on Mar. 2, 2022.

(51) Int. Cl. A61K 38/44; A61K 9/06; A61K 35/745; A61K 35/747; A61K 45/06; A61K 47/12; A61K 47/26; A61K 47/36; A61P 1/00; A61P 37/06

(52) U.S. Cl. CPC A61K 38/446; A61K 9/06; A61K 35/745; A61K 35/747; A61K 45/06; A61K 47/12; A61K 47/26; A61K 47/36; A61P 1/00; A61P 37/06; C12Y 115/01001

(57) ABSTRACT

Provided are compositions comprising super-oxide dismutase and a soluble fiber. The compositions may additionally comprise other antioxidants, sweeteners, probiotics, vitamins and nutrients. The compositions can be used as dietary supplements and for improving health and well-being. Also provided are methods of using such compounds.

### FIELD OF THE INVENTION

The present disclosure relates generally to compositions comprising super-oxide dismutase and a soluble fiber. The compositions can be used as dietary supplements and for improving health and well-being. The present disclosure further relates to methods of using compositions comprising super-oxide dismutase and a soluble fiber.

### BACKGROUND OF THE INVENTION

Superoxide dismutases (SODs) are a group of metalloenzymes that protect against cellular damage by reactive oxygen species (ROS). SOD catalyzes the dismutation of superoxide anion free radical (O₂⁻) into molecular oxygen and hydrogen peroxide (H₂O₂). ROS in cells can damage nucleic acids, proteins and lipids leading to decreased cellular function and possible apoptosis. Thus, the ability to convert ROS into harmless molecules is crucial for protecting cellular function and overall health.

While almost all organisms naturally produce some type of SOD, the levels of SOD produced in cells falls as the subject ages or when the subject is afflicted with certain health disorders. Also, the presence of various pollutants and toxins in the environment can cause increased levels of ROS in cells. Dietary supplementation of naturally produced SOD can therefore be important to maintaining health.

Another important dietary component is soluble fiber. Soluble fiber absorbs water and forms a gel in the digestive tract. Soluble fiber has many benefits in the gut, including slowing the digestion of certain types of lipids and carbohydrates, helping to prevent uptake of dietary cholesterol and preventing spikes in blood sugar levels. Soluble fiber is also important for maintaining a health gut microbiome, as gut bacteria can ferment some types of soluble fiber. Modern diets are typically low in soluble fiber and supplementation is often needed for improved health.

Diabetes mellitus (diabetes) is a common disorder of carbohydrate metabolism. In the US more than 30 million people have diabetes. In the last 20 years, the number of adults diagnosed with diabetes has more than tripled as the American population has aged and become more overweight or obese. In people who have diabetes, the normal ability of the body to utilize glucose is compromised, leading to increased blood glucose levels. Diabetes is associated with enhanced risks of cardiovascular or circulatory diseases or disorders.

Fiber, such as soluble fiber, in food has been shown to blunt the glycemic response to a meal. When fermented by gut bacteria, fiber also produces metabolites of Short Chain Fatty Acids [SCFAs] that participate in glucose homeostasis. However, high fiber food can cause stomach discomfort as can certain fiber supplements.

Antioxidants have been shown to have beneficial effects in the human body in combating free radicals, which are involved in many disease processes. However, the absorption of the effective metabolites of many antioxidants, such as polyphenols, depends on microbial metabolism of the antioxidants in the gut. Thus, for many nutritional supplements, these effective polyphenol metabolites are not produced and the antioxidant activity the supplement is limited.

### BRIEF SUMMARY OF THE INVENTION

One aspect of the present disclosure provides a liquid composition comprising: a) from about 0.03 units/mL to about 0.5 units/mL superoxide dismutase; b) from about 1.3 mg/mL to about 23 mg/mL soluble fiber; and c) water.

In embodiments, the liquid composition comprises from about 0.05 units/mL to about 0.4 units/mL superoxide dismutase. In embodiments, the liquid composition comprises from about 0.2 units/mL to about 0.3 units/mL superoxide dismutase.

In embodiments, the liquid composition comprises from about 2.7 mg/mL to about 12 mg/mL soluble fiber. In embodiments, the liquid composition comprises from about 5.55 mg/mL to about 11.11 mg/mL soluble fiber.

In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the liquid composition, the ratio of superoxide dismutase to soluble fiber is from about 1:100 to about 1:1000 by weight. In embodiments, the ratio of superoxide dismutase to soluble fiber is from about 1:500 to about 1:700 by weight.

In embodiments of the liquid composition, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments, the liquid composition further comprises from about 0.1 mg/mL to about 1.5 mg/mL of a simple sugar. In embodiments, the liquid composition further comprises from about 0.1 mg/mL to about 1.5 mg/mL d-ribose. In embodiments, the liquid composition further comprises from about 0.40 mg/mL to about 0.85 mg/mL d-ribose.

In embodiments, the liquid composition further comprises from about 1.3 mg/mL to about 9.0 mg/mL of a sugar alcohol. In embodiments, the liquid composition further comprises from about 1.3 mg/mL to about 9.0 mg/mL erythritol. In embodiments, the liquid composition further comprises from about 2.7 mg/mL to about 5.6 mg/mL erythritol.

In embodiments, the liquid composition further comprises from about 0.1 mg/mL to about 1.5 mg/mL of a pH adjusting agent. In embodiments, the liquid composition further comprises from about 0.1 mg/mL to about 1.5 mg/mL citric acid. In embodiments, the liquid composition further comprises from about 0.4 mg/mL to about 0.7 mg/mL citric acid.

In embodiments, the liquid composition further comprises from about 0.05 mg/mL to about 0.75 mg/mL of a sweetener. In embodiments, the liquid composition further comprises from about 0.05 mg/mL to about 0.75 mg/mL steviol glycoside. In embodiments, the liquid composition further comprises from about 0.2 mg/mL to about 0.35 mg/mL steviol glycoside.

In embodiments, the liquid composition further comprises a flavoring.

Another aspect of the present disclosure provides a composition comprising: a) from about 10 units to about 200 units superoxide dismutase; b) from about 500 mg to about 8000 mg soluble fiber; and c) a probiotic.

In embodiments, the probiotic composition comprises from about 50 units to about 150 units superoxide dismutase. In embodiments, the probiotic composition comprises from about 70 units to about 100 units superoxide dismutase.

In embodiments, the probiotic composition comprises from about 1000 mg to about 5000 mg soluble fiber. In embodiments, the probiotic composition comprises from about 2000 mg to about 4000 mg soluble fiber.

In embodiments of the probiotic composition, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the probiotic composition, the ratio of superoxide dismutase to soluble fiber is from about 1:100 to about 1:1000 by weight. In embodiments, the ratio of superoxide dismutase to soluble fiber is from about 1:500 to about 1:700 by weight.

In embodiments of the probiotic composition, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments of the probiotic composition, the probiotic comprises bacteria of the genus Bifidobacterium. In embodiments, the probiotic comprises bacteria of the genus Lactobacillus. In embodiments, the probiotic comprises Firmicutes lactobacillus, Actinobacteria Bifidobacteriaceae or combinations thereof.

In embodiments of the probiotic composition, the composition is in the form of a gel. In embodiments, the composition is in the form of a liquid. In embodiments, the composition is in the form of a powder.

Another aspect of the present disclosure provides a method of increasing T-cell activation in a subject comprising orally administering to the subject a composition comprising: a) from about 10 units to about 200 units superoxide dismutase; and b) from about 500 mg to about 8000 mg soluble fiber; wherein, following administration of the composition, the activation of T cells is increased in the subject. In embodiments of the method, the composition is administered in combination with an anti-cancer agent. In embodiments of the method, the composition is administered in combination with an anti-viral agent.

Another aspect of the present disclosure provides a method of increasing the production of short chain fatty acids (SCFAs) in the digestive tract of a subject comprising orally administering to the subject a composition comprising: a) from about 10 units to about 200 units superoxide dismutase; and b) from about 500 mg to about 8000 mg soluble fiber; wherein, following administration of the composition, the production of SCFAs is increased in the digestive tract of the subject. In embodiments of the method, the SCFAs increased in production are acetate, propionate, butyrate, or lactate SCFAs, or combinations thereof. In embodiments of the method, the SCFAs are increased in a manner that provides about the same ratio of acetate, propionate, butyrate, and lactate SCFAs compared to the ratio of acetate, propionate, butyrate, and lactate SCFAs prior to the increase.

Another aspect of the present disclosure provides a method of increasing the amount of bacteria of the genus Bifidobacterium or Lactobacillus in the digestive tract of a subject comprising orally administering to the subject a composition comprising: a) from about 10 units to about 200 units superoxide dismutase; and b) from about 500 mg to about 8000 mg soluble fiber; wherein, following administration of the composition, the amount of bacteria of the genus Bifidobacterium, Lactobacillus, or combinations thereof, is increased in the digestive tract of the subject. In embodiments of the method, the bacteria of the genus Bifidobacterium comprise the species Actinobacteria Bifidobacteriaceae. In embodiments of the method, the bacteria of the genus Lactobacillus comprise the species Firmicutes lactobacillus.

In embodiments of any of the methods herein, the composition comprises from about 50 units to about 150 units superoxide dismutase. In embodiments, the composition comprises from about 70 units to about 100 units superoxide dismutase.

In embodiments of any of the methods herein, the composition comprises from about 1000 mg to about 5000 mg soluble fiber. In embodiments, the composition comprises from about 2000 mg to about 4000 mg soluble fiber.

In embodiments of any of the methods herein, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of any of the methods herein, the ratio of superoxide dismutase to soluble fiber in the composition is from about 1:100 to about 1:1000 by weight. In embodiments, the ratio of superoxide dismutase to soluble fiber in the composition is from about 1:500 to about 1:700 by weight.

In embodiments of any of the methods herein, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments of any of the methods herein, the composition is in the form of a gel. In embodiments, the composition is in the form of a liquid. In embodiments, the composition is in the form of a powder.

### BRIEF DESCRIPTION OF DRAWINGS

FIG. 1 is a plot of absorbance at 490 nm (representing cell viability as described in Example 1) for Jurkat cells treated with Lipopolysaccharide (LPS), different components of the gel formulation of Example 1, the Combination (finished formulation) of the gel, and a negative control (No Treatment).

FIG. 2 is a plot showing T Cell activation as measured by CD69 and CD3 expression as described in Example 1. Jurkat cells are abbreviated JRK.

FIG. 3 is a plot showing the effect of the gel of Example 1 and its components on CD4+ differentiation in Jurkat Cells at the following concentrations: Fibersol prebiotic fiber 1.3 mg/mL; superoxide dismutase (SOD) 0.2 mg/mL; LPS 10 μg/mL; fruit juice 4.6 mg/mL and the Combination (Finished Product) 5.6 mg/mL.

FIG. 4 is a plot showing the effect of the gel of Example 1 and its components on CD8+ differentiation in Jurkat Cells at the following concentrations: Fibersol prebiotic fiber 1.3 mg/mL; superoxide dismutase (SOD) 0.2 mg/mL; LPS 10 μg/mL; fruit juice 4.6 mg/mL and the Combination (Finished Product) 5.6 mg/mL.

FIG. 5 is a plot showing the effect of the gel of Example 1 and its components on attenuation of 8-Isoprostane secretion of LPS activated Jurkat Cells as described in Example 1.

FIG. 6 is a plot showing the effect of the gel of Example 1 and its components on attenuation of Cyclooxygenase 2 (COX-2) secretion of LPS activated Jurkat Cells as described in Example 1.

FIG. 7 is a plot showing the effect of the gel of Example 1 and its components on attenuation of Interferon-gamma (IFN-γ) secretion of LPS activated Jurkat Cells as described in Example 1.

FIG. 8 is a plot showing the effect of the gel of Example 1 and its components on attenuation of Interleukin-6 (IL-6) secretion of LPS activated Jurkat Cells as described in Example 1.

FIG. 9 is a plot showing the effect of the gel of Example 1 and its components on attenuation of Transforming Growth Factor-Beta (TGF-β) secretion of LPS activated Jurkat Cells as described in Example 1.

FIG. 10 is a plot showing the effect of the gel of Example 1 and its components on attenuation of Tumor Necrosis Factor-alpha (TNF-α) secretion of LPS activated Jurkat Cells as described in Example 1.

FIG. 11 is a plot showing the effect of the gel of Example 1 and its components on attenuation of C-X-C Motif Chemokine Ligand 10 (CXCL10) secretion of LPS activated Jurkat Cells as described in Example 1.

FIG. 12 is a plot of bacterial density over time for cultures grown on goat gut treated with the complete gel product (REVIVIFY® Finished Product) or components of the gel.

FIG. 13 is a plot of bacterial density over time for cultures grown on goat gut treated with the complete gel product (REVIVIFY® Finished Product) or components of the gel.

FIG. 14A is a plot of the concentration of the short chain fatty acids (SCFAs) acetate, propionate, butyrate and lactate is in vitro gut microbial cultures when treated with the complete gel product (Combination) or components of the gel as described in Example 3.

FIG. 14B is a plot of the percent of the short chain fatty acids (SCFAs) acetate, propionate, butyrate and lactate is in vitro gut microbial cultures when treated with the complete gel product (Combination) or components of the gel as described in Example 3.

FIG. 15 is a plot showing the microbial profile of in vitro gut microbial cultures when treated with the complete gel product (Combination) or components of the gel as described in Example 3.

FIG. 16 is a plot of the concentration of malondialdehyde (MDA) biomarker secreted by Human Brain Microvascular Endothelial Cells (HBMECs) when treated with completed (Finished) gel products or components as described in Example 4.

FIG. 17 is a plot of the concentration of 4-hydroxynonenal (HINE) biomarker secreted by HBMECs when treated with completed (Finished) gel products or components as described in Example 4.

FIG. 18 is a plot of the concentration of protein carbonyls (PC) biomarkers secreted by HBMECs when treated with completed (Finished) gel products or components as described in Example 4.

FIG. 19 is a plot of the concentration of 3-nitrotyrosine (NT) biomarkers secreted by HBMECs when treated with completed (Finished) gel products or components as described in Example 4.

### DETAILED DESCRIPTION OF THE INVENTION

The present disclosure provides compositions comprising superoxide dismutase and a soluble fiber. The compositions disclosed herein can be used as supplements to increase the levels of superoxide dismutase and soluble fiber in subject. As described herein, the compositions can also comprise additional components such as antioxidants, vitamins or other nutrients along with excipients and other formulation agents.

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict from any reference cited herein and the specific teachings of this specification shall be resolved in favor of the latter. Likewise, any conflict from an art-understood definition of a word or phrase and a definition of the word or phrase as specifically taught in this specification shall be resolved in favor of the latter.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

Technical and scientific terms used herein have the meaning commonly understood by one of skill in the art to which the present application pertains, unless otherwise defined. Reference is made herein to various methodologies and materials known to those of skill in the art.

The term “superoxide dismutase” (sometimes abbreviated SOD) as used herein refers an enzyme that catalyzes the dismutation of superoxide anion free radical (O₂⁻) into molecular oxygen and hydrogen peroxide (H₂O₂). Superoxide dismutase has the Enzyme Classification (EC) number 1.15.1.1. Superoxide dismutase is an important component in protecting cells against damage from reactive oxygen species (ROS) such as O₂⁻ that are formed during cellular metabolism of oxygen. Superoxide dismutase thus acts as an important antioxidant and is present in almost all types of cellular organisms. However, particularly in complex organisms such as mammals, cellular levels of superoxide dismutase decrease as the organism ages, making the organism more susceptible to cellular damage from reactive oxygen species.

The term “soluble fiber” as used herein refers to any type of water soluble dietary fiber. When consumed, soluble fiber absorbs water to form a gel in the gut of the organism that helps to slow the metabolism of lipids and carbohydrates. Soluble fiber is also a prebiotic that can be fermented by gut bacteria which helps maintain a healthy gut microbiome.

The term “antioxidant” as used herein refers to a substance that significantly decreases the adverse effects of reactive species, such as reactive oxygen and nitrogen species by wholly or partially neutralizing reactive species. Antioxidants can be classified as “primary antioxidants” and “secondary antioxidants.” Primary antioxidants delay or inhibit the initiation step of oxidation, while secondary antioxidants slow down the oxidation by removing the substrate or by quenching free oxygen radicals.

### 1) Superoxide Dismutase

The embodiments, the compositions disclosed herein comprise an amount of superoxide dismutase as measured in units of activity per mg of protein. For enzyme activity, one unit (U) (expressed in mol/min) is defined as the amount of the enzyme that catalyzes the conversion of one micromole of substrate per minute under specified conditions. The units of superoxide dismutase activity can be measured by any known method. For example, methods of determining superoxide dismutase activity in units are described in McCord, J. M. and Fridovich, I., J Biol. Chem. 1969, 244:6049-6055; Weydert et al., Nature Protocols 2010, 5(1): 51-66; and in the technical protocol at https://www.sigmaaldrich.com/technical-documents/protocols/biology/enzymatic-assay-of-superoxide-dismutase.html; the disclosures of each of which are incorporated by reference herein. In embodiments, a unit of superoxide dismutase activity is defined as the amount of superoxide dismutase that will inhibit the rate of reduction of cytochrome c by 50% in a coupled system, using xanthine and xanthine oxidase at pH 7.8 at 25° C. in a 3.0 ml reaction volume.

In embodiments, the concentration of the amount of superoxide dismutase in the compositions disclosed herein is measured in the total amount of units of superoxide dismutase in the composition. In embodiments, the composition comprises from about 10 units to about 200 units superoxide dismutase. In embodiments, the composition comprises comprising from about 50 units to about 150 units superoxide dismutase. In embodiments, the composition comprises from about 70 units to about 100 units superoxide dismutase. In embodiments, the composition comprises from about 20 units to about 190 units superoxide dismutase. In embodiments, the composition comprises from about 30 units to about 180 units superoxide dismutase. In embodiments, the composition comprises from about 40 units to about 170 units superoxide dismutase. In embodiments, the composition comprises from about 50 units to about 160 units superoxide dismutase. In embodiments, the composition comprises from about 60 units to about 150 units superoxide dismutase. In embodiments, the composition comprises from about 70 units to about 140 units superoxide dismutase. In embodiments, the composition comprises from about 80 units to about 130 units superoxide dismutase. In embodiments, the composition comprises from about 90 units to about 130 units superoxide dismutase. In embodiments, the composition comprises from about 75 units to about 95 units superoxide dismutase. In embodiments, the composition comprises from about 80 units to about 90 units superoxide dismutase. In embodiments, the composition comprises about 20, 30, 40, 50, 60, 70, 80, 90, 100, 110, 120, 130, 140, 150, or 160 units superoxide dismutase. In embodiments, the composition comprises about 84 units superoxide dismutase.

The superoxide dismutase used in the compositions disclosed herein can be obtained from any source of the enzyme. In embodiments, the superoxide dismutase is extracted from melon, bovine liver, heterotrophic bacteria or marine phytoplankton. The superoxide dismutase can also be any type of the enzyme. In embodiments, the superoxide dismutase is a copper/zinc superoxide dismutase, an iron/manganese superoxide dismutase or a nickel superoxide dismutase.

In embodiments, the superoxide dismutase is extracted from a plant. In embodiments, the plant is a fruit, a grain or a tuber. In embodiments, the plant is a fruit selected from melon, citrus fruit, peach, pear, apple or banana. In embodiments, the plant is a grain selected from wheat, barley, rye, millet, oat, spelt, bulger, sorghum and farro. In embodiments, the plant is a tuber selected from horseradish, potato, yam, sweet potato, cassava or dahlia.

In embodiments, the superoxide dismutase is extracted from an animal. In embodiments, the animal is a cow, pig, sheep or goat.

In embodiments, the superoxide dismutase is extracted from a microorganism. In embodiments, the microorganism is phytoplankton or bacteria. In embodiments, the microorganism is a heterotrophic bacteria, e.g., a bacteria that takes the sugars it needs for energy production from their environment.

### 2) Soluble Fiber

The ratio of superoxide dismutase to soluble fiber can be adjusted as needed by varying the amount of either or both components of the composition. In embodiments, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

Without wishing to be bound by theory, the inventor has found that the presence of soluble fiber together with superoxide dismutase at specific ratios synergistically allows for improved stability and uptake in the digestive tract (gut) as demonstrated in the examples below. One mechanism behind this improved stability and uptake is that the soluble fiber forms a gel in the digestive tract and that this formed gel entraps and protects the superoxide dismutase while also allowing it to be more readily absorbed through the wall of the gut. The soluble fiber and the antioxidants formulations of the present disclosure provide synergistic effects in that the soluble prebiotic fiber acts as a fertilizer for the bacteria in the colon, while the polyphenols are absorbed in a substantial part in the colon and subject to extensive catabolism by colonic microbiota. The presence of the prebiotic fiber enhances the action of the colonic microbiota, thereby leading to more effective absorption of the polyphenols in the colon. A large majority of polyphenols from an antioxidant supplement end up in the large intestine where they undergo microbial metabolism into their active metabolites where they can exert an antioxidant effect. The soluble fiber modulates the gut microbes and maximizes polyphenol metabolism, producing many antioxidant, anti-inflammatory, and anti-infection effects.

In embodiments, the soluble fiber is a prebiotic fiber. As used herein a “prebiotic fiber” is a soluble fiber that forms a matrix in the digestive tract that provides a substrate for the propagation of microorganisms in the gut.

In embodiments of the compositions disclosed herein, various types of soluble fibers can be used, including mixtures of two, three, four, five, six or more different types of soluble fibers. In embodiments, the soluble fiber is a water soluble polysaccharide. In embodiments, the soluble fiber is isolated from corn, wheat, barley, rye, beans, apples, pears, peaches, citrus fruits, berries, peas, rice bran or oats. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble fiber is digestion-resistant maltodextrin. In embodiments, the soluble fiber is Fibersol-2® as sold by Archer Daniels Midland Company and Matsutani Chemical Industry Co., Ltd. In embodiments, the soluble fiber an alternative type of Fibersol-2® such as Fibersol-2AG, Fibersol-LQ, Fibersol-2L, Fibersol-DLQ or non-GMO Fibersol.

In some embodiments, the soluble fiber is corn-based digestion resistant maltodextrin (Fibersol-2) prebiotic fiber.

### 3) Liquid Compositions

In embodiments, the present disclosure provides a liquid composition comprising superoxide dismutase, soluble fiber, and water. In embodiments, the present disclosure provides a liquid composition comprising: a) from about 0.03 units/mL to about 0.5 units/mL superoxide dismutase; b) from about 1.3 mg/mL to about 23 mg/mL soluble fiber; and c) water.

In embodiments, the liquid composition comprises from about 0.05 units/mL to about 0.4 units/mL superoxide dismutase. In embodiments, the liquid composition comprises from about 0.2 units/mL to about 0.3 units/mL superoxide dismutase. In embodiments, the liquid composition comprises from about 0.1 units/mL to about 0.4 units/mL superoxide dismutase. In embodiments, the liquid composition comprises from about 0.15 units/mL to about 0.35 units/mL superoxide dismutase. In embodiments, the liquid composition comprises about 0.05, 0.1, 0.15, 0.2, 0.25, 0.3, 0.35, or 0.4 units/mL superoxide dismutase. In embodiments, the liquid composition comprises about 0.20, 0.21, 0.22, 0.23, 0.24, 0.25, 0.26, 0.27, 0.28, 0.29 or 0.30 units/mL superoxide dismutase.

In embodiments, the liquid composition comprises from about 2.7 mg/mL to about 12 mg/mL soluble fiber. In embodiments, the liquid composition comprises from about 5.55 mg/mL to about 11.11 mg/mL soluble fiber. In embodiments, the liquid composition comprises from about 2 mg/mL to about 15 mg/mL soluble fiber. In embodiments, the liquid composition comprises from about 4 mg/mL to about 12 mg/mL soluble fiber. In embodiments, the liquid composition comprises from about 5 mg/mL to about 12 mg/mL soluble fiber. In embodiments, the liquid composition comprises from about 6 mg/mL to about 12 mg/mL soluble fiber. In embodiments, the liquid composition comprises from about 7 mg/mL to about 12 mg/mL soluble fiber. In embodiments, the liquid composition comprises from about 8 mg/mL to about 12 mg/mL soluble fiber. In embodiments, the liquid composition comprises from about 9 mg/mL to about 12 mg/mL soluble fiber. In embodiments, the liquid composition comprises from about 10 mg/mL to about 12 mg/mL soluble fiber. In embodiments, the liquid composition comprises about 2.78, 5.56, 8.33, 11.11 or 13.89 mg/mL soluble fiber.

In embodiments, the liquid composition comprises any of the superoxide dismutases disclosed herein. In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the liquid composition, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

In embodiments, the liquid composition comprises any of the soluble fibers disclosed herein. In embodiments, the soluble fiber is a prebiotic fiber. In embodiments, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments, the liquid compositions can further comprise additional ingredients, including sweeteners, pH adjusting agents, flavors and other agents, including combinations of these agents.

In embodiments, the liquid composition comprises a sweetener. In embodiments, the liquid composition comprises a combination of sweeteners, examples of which are provided below.

In embodiments, the sweetener is a simple sugar. In embodiments, the simple sugar is ribose, glucose, fructose, sucrose, lactose or combinations thereof. In embodiments, the simple sugar is ribose. In embodiments, the simple sugar is d-ribose.

In embodiments, the liquid composition comprises from about 0.1 mg/mL to about 1.5 mg/mL of a simple sugar. In embodiments, the liquid composition comprises from about 0.2 mg/mL to about 1.3 mg/mL of a simple sugar. In embodiments, the liquid composition comprises from about 0.3 mg/mL to about 1.0 mg/mL of a simple sugar. In embodiments, the liquid composition comprises from about 0.4 mg/mL to about 0.8 mg/mL of a simple sugar. In embodiments, the liquid composition comprises from about 0.4 mg/mL to about 0.85 mg/mL of a simple sugar. In embodiments, the liquid composition comprises from about 0.4 mg/mL to about 0.6 mg/mL of a simple sugar. In embodiments, the liquid composition comprises from about 0.4, 0.45, 0.5, 0.55, 0.6, 0.65, 0.7, 0.75, 0.8, or 0.85 mg/mL of a simple sugar.

In embodiments, the liquid composition comprises from about 0.1 mg/mL to about 1.5 mg/mL of d-ribose. In embodiments, the liquid composition comprises from about 0.2 mg/mL to about 1.3 mg/mL of d-ribose. In embodiments, the liquid composition comprises from about 0.3 mg/mL to about 1.0 mg/mL of d-ribose. In embodiments, the liquid composition comprises from about 0.4 mg/mL to about 0.8 mg/mL of d-ribose. In embodiments, the liquid composition comprises from about 0.4 mg/mL to about 0.85 mg/mL of d-ribose. In embodiments, the liquid composition comprises from about 0.4 mg/mL to about 0.6 mg/mL of d-ribose. In embodiments, the liquid composition comprises from about 0.4, 0.45, 0.5, 0.55, 0.6, 0.65, 0.7, 0.75, 0.8, or 0.85 mg/mL of d-ribose.

In embodiments of the liquid composition, the sweetener comprises a sugar alcohol. In embodiments, the sugar alcohol is erythritol, mannitol, sorbitol, xylitol, lactitol, isomalt or combinations thereof. In embodiments, the sugar alcohol is erythritol.

In embodiments, the liquid composition comprises from about 1.3 mg/mL to about 9.0 mg/mL of a sugar alcohol. In embodiments, the liquid composition comprises from about 2.0 mg/mL to about 8.0 mg/mL of a sugar alcohol. In embodiments, the liquid composition comprises from about 3.9 mg/mL to about 7.0 mg/mL of a sugar alcohol. In embodiments, the liquid composition comprises from 2.7 mg/mL to about 5.6 mg/mL of a sugar alcohol. In embodiments, the liquid composition comprises about 2.0, 2.5, 3.0, 3.5, 4.0, 4.15, 4.5, 5.0, 5.5, 6.0, 6.5, 7.0, 7.5 or 8.0 mg/mL of a sugar alcohol.

In embodiments, the liquid composition comprises from about 1.3 mg/mL to about 9.0 mg/mL of erythritol. In embodiments, the liquid composition comprises from about 2.0 mg/mL to about 8.0 mg/mL of erythritol. In embodiments, the liquid composition comprises from about 3.9 mg/mL to about 7.0 mg/mL of erythritol. In embodiments, the liquid composition comprises from 2.7 mg/mL to about 5.6 mg/mL of erythritol. In embodiments, the liquid composition comprises about 2.0, 2.5, 3.0, 3.5, 4.0, 4.15, 4.5, 5.0, 5.5, 6.0, 6.5, 7.0, 7.5 or 8.0 mg/mL of erythritol.

In embodiments, the sweetener is a steviol glycoside. In embodiments, the steviol glycoside is Rebaudioside A, Stevioside, Rebaudioside C, Dulcoside A, Rebaudioside B, Rebaudioside D, Rebaudioside E, steviolbioside or combinations thereof.

In embodiments, the liquid composition comprises from about 0.05 mg/mL to about 0.75 mg/mL of steviol glycoside. In embodiments, the liquid composition comprises from about 0.2 mg/mL to about 0.35 mg/mL steviol glycoside. In embodiments, the liquid composition comprises from about 0.1 mg/mL to about 0.5 mg/mL steviol glycoside. In embodiments, the liquid composition comprises from about 0.2 mg/mL to about 0.3 mg/mL steviol glycoside. In embodiments, the liquid composition comprises about 0.10, 0.15, 0.20, 0.25, 0.27, 0.30, 0.35, 0.40, 0.45 or 0.50 mg/mL steviol glycoside.

In embodiments, the liquid composition comprises from about 0.05 mg/mL to about 0.75 mg/mL of Rebaudioside A. In embodiments, the liquid composition comprises from about 0.2 mg/mL to about 0.35 mg/mL Rebaudioside A. In embodiments, the liquid composition comprises from about 0.1 mg/mL to about 0.5 mg/mL Rebaudioside A. In embodiments, the liquid composition comprises from about 0.2 mg/mL to about 0.3 mg/mL Rebaudioside A. In embodiments, the liquid composition comprises about 0.10, 0.15, 0.20, 0.25, 0.27, 0.30, 0.35, 0.40, 0.45 or 0.50 mg/mL Rebaudioside A.

In embodiments, the liquid composition further comprises a pH adjusting agent. In embodiments, the pH adjusting agent is any food-safe agent that can be used to change the pH of the liquid composition. In embodiments, the pH adjusting agent is citric acid, acetic acid, hydrochloric acid, lactic acid, malic acid, phosphoric acid, tartaric acid or combinations thereof.

In embodiments, the liquid composition comprises from about 0.1 mg/mL to about 1.5 mg/mL of a pH adjusting agent. In embodiments, the liquid composition comprises from about 0.2 mg/mL to about 1.3 mg/mL of a pH adjusting agent. In embodiments, the liquid composition comprises from about 0.3 mg/mL to about 1.0 mg/mL of a pH adjusting agent. In embodiments, the liquid composition comprises from about 0.4 mg/mL to about 0.8 mg/mL of a pH adjusting agent. In embodiments, the liquid composition comprises from about 0.4 mg/mL to about 0.85 mg/mL of a pH adjusting agent. In embodiments, the liquid composition comprises from about 0.4 mg/mL to about 0.6 mg/mL of a pH adjusting agent. In embodiments, the liquid composition comprises from about 0.4, 0.45, 0.5, 0.55, 0.6, 0.65, 0.7, 0.75, 0.8, or 0.85 mg/mL of a pH adjusting agent.

In embodiments, the liquid composition comprises from about 0.1 mg/mL to about 1.5 mg/mL of citric acid. In embodiments, the liquid composition comprises from about 0.2 mg/mL to about 1.3 mg/mL of citric acid. In embodiments, the liquid composition comprises from about 0.3 mg/mL to about 1.0 mg/mL of citric acid. In embodiments, the liquid composition comprises from about 0.4 mg/mL to about 0.8 mg/mL of citric acid. In embodiments, the liquid composition comprises from about 0.4 mg/mL to about 0.85 mg/mL of citric acid. In embodiments, the liquid composition comprises from about 0.4 mg/mL to about 0.6 mg/mL of citric acid. In embodiments, the liquid composition comprises from about 0.4, 0.45, 0.5, 0.55, 0.6, 0.65, 0.7, 0.75, 0.8, or 0.85 mg/mL of citric acid.

In embodiments, the liquid composition comprises a flavoring. In embodiments, the flavor is a natural flavoring. In embodiments, the flavor is an artificial flavoring. In embodiments, the flavor if a fruit juice flavoring. In embodiments, the flavoring is pomegranate, red grape, blueberry, dark sweet cherry, sour cherry, goji berry, acai berry, blackberry, raspberry, strawberry, gooseberry, cranberry, orange, grapefruit, watermelon, beet, apple, lemon, lime, lychee, pineapple, prune, mango or combinations thereof. In embodiments, the liquid composition comprises a cola flavoring.

In embodiments, the liquid composition is formulated in a beverage. In embodiments, the liquid composition is formulated as a hydration beverage, a protein shake, juice, tea, coffee, milk, kefir, ice cream, yogurt, a smoothie, broth or soup.

In embodiments, the liquid composition has a volume of from about 15 mL to about 1500 mL. In embodiments, the liquid composition has a volume of from about 30 mL to about 1200 mL. In embodiments, the liquid composition has a volume of from about 50 mL to about 1000 mL. In embodiments, the liquid composition has a volume of from about 100 mL to about 500 mL. In embodiments, the liquid composition has a volume of from about 200 mL to about 400 mL. In embodiments, the liquid composition has a volume of from about 200 mL to about 1000 mL. In embodiments, the liquid composition has a volume of from about 300 mL to about 1000 mL. In embodiments, the liquid composition has a volume of about 50, 100, 150, 200, 250, 300, 350, 400, 450, 500, 550, 600, 650, 700, 750, 800, 850, 900, 950 or 1000 mL.

In embodiments, the liquid composition is packaged in a bottle. In embodiments, the bottle is a glass bottle. In embodiments, the bottle is a plastic bottle. In embodiments, the liquid composition is packaged in a can. In embodiments, the liquid composition is package in a drink box.

In embodiments, a specific example of a liquid composition is provided in Table 1:

TABLE 1

| 1. | SOD [melon extract powder, | 6.0 mg | |
|---|---|---|---|
| | 14,000 units/g] | [84 enzyme units] | |
| 2. | Fibersol-2 | 4000.0 mg | |
| | | [i may reduce to 3 or 2 g] | |
| 3. | D-Ribose- | 200.0 | mg |
| 4. | Erythritol | 1494.0 | mg |
| 5. | Citric acid | 200.0 | mg |
| 6. | Rebaudioside A- | 100.0 | mg |
| 7. | Flavor | 100.0 | mg |
| 8. | Water qs- | 360 | ml. |

### 4) Powder Compositions

In some embodiments of the compositions disclosed herein, the composition is in the form of a powder. In embodiments where the composition is in the form of a powder, it can be consumed in dry powder form or added to a beverage or food. In embodiments, the powder is mixed into water, a hydration beverage, a protein shake, juice, tea, coffee, milk, kefir, ice cream, yogurt, a smoothie, broth or soup prior to consumption.

In embodiments, the powder composition comprises a high concentration of superoxide dismutase by weight, e.g., greater than 0.1 ppm, greater than 0.5 ppm, greater than 1 ppm, greater than 2 ppm, greater than 5 ppm, greater than 10 ppm, greater than 20 ppm, greater than 50 ppm, greater than 100 ppm, greater than 200 ppm, greater than 300 ppm, greater than 400 ppm or greater than 500 ppm. In embodiments, the powder composition comprises from about 0.1 ppm to about 10 ppm superoxide dismutase by weight. In embodiments, the powder composition comprises from about 0.5 ppm to about 5 ppm superoxide dismutase by weight. In embodiments, the powder composition comprises from about 0.7 ppm to about 2 ppm superoxide dismutase by weight. In embodiments, the powder composition comprises from about 0.8 ppm to about 1.2 ppm superoxide dismutase by weight. In embodiments, the powder composition comprises about 0.1, 0.25, 0.5, 0.75, 1.0, 1.1, 1.2, 1.25, 1.3, 1.4, 1.5, 1.6, 1.7, 1.75, 1.8, 1.9, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, or 10.0 ppm superoxide dismutase by weight.

In embodiments, the powder composition comprises from about 5 ppm to about 15 ppm superoxide dismutase by weight. In embodiments, the powder composition comprises from about 7.5 ppm to about 12.5 ppm superoxide dismutase by weight. In embodiments, the powder composition comprises from about 8 ppm to about 11 ppm superoxide dismutase by weight. In embodiments, the powder composition comprises from about 9 ppm to about 11 ppm superoxide dismutase by weight. In embodiments, the powder composition comprises about 5, 6, 7, 8, 9, 10, 11, 12, 13, 14 or 15 ppm superoxide dismutase by weight.

In embodiments, the powder composition comprises from about 1% to about 90% soluble fiber by weight. In embodiments, the powder composition comprises from about 1% to about 50% soluble fiber by weight. In embodiments, the powder composition comprises from about 5% to about 25% soluble fiber by weight. In embodiments, the powder composition comprises from about 10% to about 20% soluble fiber by weight. In embodiments, the powder composition comprises from about 12% to about 14% soluble fiber by weight. In embodiments, the power composition comprises about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% soluble fiber by weight.

In embodiments, the powder composition comprises from about 50% to about 75% soluble fiber by weight. In embodiments, the powder composition comprises from about 50% to about 70% soluble fiber by weight. In embodiments, the powder composition comprises from about 60% to about 70% soluble fiber by weight. In embodiments, the powder composition comprises from about 50% to about 80% soluble fiber by weight. In embodiments, the power composition comprises about 50%, 55%, 60%, 65%, 66%, 67%, 70%, 75%, 80%, 85% or 90% soluble fiber by weight.

In embodiments, the powder composition comprises a sweetener. In embodiments, the powder composition comprises a combination of sweeteners, examples of which are provided below.

In embodiments, the sweetener is a simple sugar. In embodiments, the simple sugar is ribose, glucose, fructose, sucrose, lactose or combinations thereof. In embodiments, the simple sugar is ribose. In embodiments, the simple sugar is d-ribose.

In embodiments, the powder composition comprises from about 1% to about 10% by weight of a simple sugar. In embodiments, the powder composition comprises from about 2% to about 8% by weight of a simple sugar. In embodiments, the powder composition comprises from about 2% to about 5% by weight of a simple sugar. In embodiments, the powder composition comprises from about 1%, 2%, 3%, 4%, 5%, 6%, 7%, 8%, 9% or 10% by weight of a simple sugar.

In embodiments, the powder composition comprises from about 1% to about 10% by weight of d-ribose. In embodiments, the powder composition comprises from about 2% to about 8% by weight of d-ribose. In embodiments, the powder composition comprises from about 2% to about 5% by weight of d-ribose. In embodiments, the powder composition comprises from about 1%, 2%, 3%, 4%, 5%, 6%, 7%, 8%, 9% or 10% by weight of d-ribose.

In embodiments of the powder composition, the sweetener comprises a sugar alcohol. In embodiments, the sugar alcohol is erythritol, mannitol, sorbitol, xylitol, lactitol, isomalt or combinations thereof. In embodiments, the sugar alcohol is erythritol.

In embodiments, the powder composition comprises from about 10% to about 50% by weight of erythritol. In embodiments, the powder composition comprises from about 15% to about 35% by weight of erythritol. In embodiments, the powder composition comprises from about 20% to about 30% by weight of erythritol. In embodiments, the powder composition comprises from about 15%, 20%, 25, 30%, 35%, 40%, 45% or 50% by weight of erythritol.

In embodiments, the sweetener is a steviol glycoside. In embodiments, the steviol glycoside is Rebaudioside A, Stevioside, Rebaudioside C, Dulcoside A, Rebaudioside B, Rebaudioside D, Rebaudioside E, steviolbioside or combinations thereof.

In embodiments, the powder composition comprises from about 0.5% to about 5% by weight of a steviol glycoside. In embodiments, the powder composition comprises from about 1% to about 4% by weight of a steviol glycoside. In embodiments, the powder composition comprises from about 1% to about 2.5% by weight of a steviol glycoside. In embodiments, the powder composition comprises from about 1%, 1.5%, 1.6% 2%, 2.5%, 3%, 3.5%, or 4% by weight of a steviol glycoside.

In embodiments, the powder composition comprises from about 0.5% to about 5% by weight of Rebaudioside A. In embodiments, the powder composition comprises from about 1% to about 4% by weight of Rebaudioside A. In embodiments, the powder composition comprises from about 1% to about 2.5% by weight of Rebaudioside A. In embodiments, the powder composition comprises from about 1%, 1.5%, 1.6% 2%, 2.5%, 3%, 3.5%, or 4% by weight of Rebaudioside A.

In embodiments, the liquid composition further comprises a pH adjusting agent. In embodiments, the pH adjusting agent is any food-safe agent that can be used to change the pH of the liquid composition. In embodiments, the pH adjusting agent is citric acid, acetic acid, hydrochloric acid, lactic acid, malic acid, phosphoric acid, tartaric acid or combinations thereof.

In embodiments, the powder composition comprises from about 1% to about 10% by weight of a pH adjusting agent. In embodiments, the powder composition comprises from about 2% to about 8% by weight of a pH adjusting agent. In embodiments, the powder composition comprises from about 2% to about 5% by weight of a pH adjusting agent. In embodiments, the powder composition comprises from about 1%, 2%, 3%, 4%, 5%, 6%, 7%, 8%, 9% or 10% by weight of a pH adjusting agent.

In embodiments, the powder composition comprises from about 1% to about 10% by weight of citric acid. In embodiments, the powder composition comprises from about 2% to about 8% by weight of citric acid. In embodiments, the powder composition comprises from about 2% to about 5% by weight of citric acid. In embodiments, the powder composition comprises from about 1%, 2%, 3%, 4%, 5%, 6%, 7%, 8%, 9% or 10% by weight of citric acid.

In embodiments, a specific example of a powder composition is provided in Table 2:

TABLE 2
Total amount: 6 grams.

| 1. | SOD [melon extract powder, | 6.0 mg | |
|---|---|---|---|
| | 14,000 units/g] | [84 enzyme units] | |
| 2. | Fibersol-2 | 4000.0 | mg |
| 3. | D-Ribose | 200.0 | mg |
| 4. | Erythritol [excipients] | 1494.0 | mg |
| 5. | Citric acid [excipients] | 200.0 | mg |
| 6. | Sweetener [Rebaudioside A], | 100.0 | mg |
| | natural stevia | | |

### 5) Gel Compositions

In embodiments of the compositions disclosed herein, the composition is in the form of a gel. In embodiments where the composition is in the form of a gel, it can be consumed in directly in that form. In other embodiments, the gel can be added to a beverage or food. In embodiments, the gel is mixed into water, a hydration beverage, a protein shake, juice, tea, coffee, milk, kefir, ice cream, yogurt, a smoothie, broth or soup prior to consumption.

In embodiments, the gel composition comprises from about 0.005 mg/mL to about 5.0 mg/mL superoxide dismutase. In embodiments, the gel composition comprises from about 0.01 mg/mL to about 2.5 mg/mL superoxide dismutase. In embodiments, the gel composition comprises from about 0.05 mg/mL to about 1.0 mg/mL superoxide dismutase. In embodiments, the gel composition comprises from about 0.1 mg/mL to about 0.5 mg/mL superoxide dismutase. In embodiments, the gel composition comprises about 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/mL superoxide dismutase.

In embodiments, the gel composition comprises from about 50 mg/mL to about 1000 mg/mL soluble fiber. In embodiments, the gel composition comprises from about 70 mg/mL to about 500 mg/mL soluble fiber. In embodiments, the gel composition comprises from about 90 mg/mL to about 250 mg/mL soluble fiber. In embodiments, the gel composition comprises from about 100 mg/mL to about 200 mg/mL soluble fiber. In embodiments, the gel composition comprises about 50, 75, 100, 110, 120, 125, 130, 133, 135, 140, 150, 160, 170, 175, 180, 190, 200, 225, 250, 275, 300, 325, 350, 375, 400, 425, 450, 475 or 500 mg/mL soluble fiber.

In embodiments of the compositions disclosed herein, the composition comprising superoxide dismutase and soluble fiber also comprises a fruit juice. The fruit juice can provide additional antioxidants, soluble fiber, insoluble fiber, vitamins and nutrients to the composition. In embodiments, the gel compositions disclosed herein comprise a fruit juice.

In embodiments, the fruit juice is pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice, prune juice or combinations thereof. In embodiments, the composition comprises two, three four, five or six types of juice selected from: pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice and prune juice.

In embodiments, the fruit juice can be concentrated, e.g., having some of the water in the original juice removed. In embodiments, the fruit juice is concentrated pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice, prune juice or combinations thereof. In embodiments, the composition comprises two, three four, five or six types of concentrated juice selected from: concentrated pomegranate juice, concentrated red grape juice, concentrated blueberry juice, concentrated dark sweet cherry juice, concentrated sour cherry juice, concentrated goji berry juice, concentrated acai berry juice, concentrated blackberry juice, concentrated raspberry juice, concentrated strawberry juice, concentrated gooseberry juice, concentrated cranberry juice, concentrated orange juice, concentrated grapefruit juice, concentrated watermelon juice, concentrated beet juice, concentrated apple juice, concentrated lemon juice, concentrated lime juice, concentrated lychee juice, concentrated pineapple juice, concentrated prune juice. In embodiments, the concentrated fruit juice has about 60% to about 97% of the water of the juice removed. In embodiments, the concentrated fruit juice has about 85% to about 95% of the water of the juice removed. In embodiments, the concentrated fruit juice has about 60%, 65%, 70%, 75%, 80%, 85%, 90%, 95%, 96%, 97%, 98% or 99% of the water of the fruit juice removed. Water can be removed from the fruit juices using any method known in the art for concentrating fruit juices.

In embodiments, the composition comprises from about 5 mg/mL to about 200 mg/mL of the concentrated fruit juice. In embodiments, the composition comprises from about 75 mg/mL to about 150 mg/mL concentrated pomegranate juice. In embodiments, the composition comprises from about 75 mg/mL to about 150 mg/mL concentrated red grape juice. In embodiments, the composition comprises from about 25 mg/mL to about 100 mg/mL concentrated blueberry juice. In embodiments, the composition comprises from about 20 mg/mL to about 80 mg/mL concentrated dark sweet cherry juice. In embodiments, the composition comprises from about 20 mg/mL to about 80 mg/mL concentrated sour cherry juice. In embodiments, the composition comprises from about 2 mg/mL to about 20 mg/mL concentrated goji berry juice. In embodiments, the composition comprises from about 2 mg/mL to about 20 mg/mL concentrated acai berry juice.

In embodiments of the gel compositions disclosed herein, the composition comprising superoxide dismutase and soluble fiber also comprises aloe vera. The aloe vera can provide additional antioxidants, soluble fiber, insoluble fiber, vitamins and nutrients to the composition.

In some embodiments, the aloe vera is concentrated aloe vera. In embodiments, the aloe vera can be concentrated, e.g., having some of the water removed from the aloe vera that is extracted from the aloe vera plant. In embodiments, the concentrated aloe vera has about 60% to about 97% of the water of the aloe vera removed. In embodiments, the concentrated aloe vera has about 60%, 65%, 70%, 75%, 80%, 85%, 90%, 95%, 96%, 97%, 98% or 99% of the water of the aloe vera removed. In embodiments, the composition comprises from about 2 mg/mL to about 20 mg/mL concentrated aloe vera.

In embodiments of the gel compositions disclosed herein, the composition comprising superoxide dismutase and soluble fiber also comprises green tea. The green tea can provide additional antioxidants, vitamins and nutrients to the composition.

In embodiments, the green tea is concentrated green tea. In embodiments, the concentrated green tea is a green tea extract made from green tea leaves or green tea powder. In embodiments, the green tea is added to the composition in powder form. In embodiments, the composition comprises from about 2 mg/mL to about 20 mg/mL concentrated green tea.

In embodiments of the gel compositions disclosed herein, the composition comprising superoxide dismutase and soluble fiber also comprises resveratrol. The resveratrol can provide additional antioxidants to the composition. In some embodiments, the composition comprises from about 0.5 mg/mL to about 6 mg/mL resveratrol.

In embodiments, a specific gel composition is provided in Table 3

TABLE 3

| SI. # | Ingredient | QTY in mg |
|---|---|---|
| 1 | Soluble corn fiber (Fibersol-2) | 4000 |
| 2 | Pomegranate Juice Concentrate | 4000 |
| 3 | Concord Grape Juice Concentrate | 4000 |
| 4 | Blueberry Juice Concentrate | 2000 |
| 5 | Dark Sweet Cherry Juice Concentrate | 1450 |
| 6 | Sour Cherry Juice Concentrate | 1450 |
| 7 | Goji Pulp | 294 |
| 8 | Acai Concentrate Juice | 200 |
| 9 | Aloe vera juice | 200 |
| 10 | Green Tea | 200 |
| 11 | Resveratrol | 80 |
| 12 | Extramel SOD (14000 unit/g) | 6 |
| 13 | CMC | 60.5 |
| 14 | Xanthan | 60.5 |
| 15 | QS Water (30 ml) | |

### 6) Probiotic Compositions

In embodiments, disclosed herein are compositions comprising superoxide dismutase, soluble fiber and a probiotic. In embodiments, the probiotic composition comprises: a) from about 10 units to about 200 units superoxide dismutase; b) from about 500 mg to about 8000 mg soluble fiber; and c) a probiotic.

In embodiments, the concentration of the amount of superoxide dismutase in the probiotic compositions disclosed herein is measured in the total amount of units of superoxide dismutase in the composition. In embodiments, the composition comprises from about 10 units to about 200 units superoxide dismutase. In embodiments, the composition comprises comprising from about 50 units to about 150 units superoxide dismutase. In embodiments, the composition comprises from about 70 units to about 100 units superoxide dismutase. In embodiments, the composition comprises from about 20 units to about 190 units superoxide dismutase. In embodiments, the composition comprises from about 30 units to about 180 units superoxide dismutase. In embodiments, the composition comprises from about 40 units to about 170 units superoxide dismutase. In embodiments, the composition comprises from about 50 units to about 160 units superoxide dismutase. In embodiments, the composition comprises from about 60 units to about 150 units superoxide dismutase. In embodiments, the composition comprises from about 70 units to about 140 units superoxide dismutase. In embodiments, the composition comprises from about 80 units to about 130 units superoxide dismutase. In embodiments, the composition comprises from about 90 units to about 130 units superoxide dismutase. In embodiments, the composition comprises from about 75 units to about 95 units superoxide dismutase. In embodiments, the composition comprises from about 80 units to about 90 units superoxide dismutase. In embodiments, the composition comprises about 20, 30, 40, 50, 60, 70, 80, 90, 100, 110, 120, 130, 140, 150, or 160 units superoxide dismutase. In embodiments, the composition comprises about 84 units superoxide dismutase.

In embodiments, the probiotic composition comprises from about 1000 mg to about 5000 mg soluble fiber. In embodiments, the probiotic composition comprises from about 2000 mg to about 4000 mg soluble fiber. In embodiments, the probiotic composition comprises from about 1000 mg to about 10000 mg soluble fiber. In embodiments, the probiotic composition comprises from about 2000 mg to about 9000 mg soluble fiber. In embodiments, the probiotic composition comprises from about 3000 mg to about 8000 mg soluble fiber. In embodiments, the probiotic composition comprises from about 2000 mg to about 4000 mg soluble fiber. In embodiments, the probiotic composition comprises about 500, 1000, 1500, 2000, 2500, 3000, 3500, 4000 4500 or 5000 mg soluble fiber.

In embodiments, the ratio of superoxide dismutase to soluble fiber in the probiotic composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

The probiotic composition can comprise one or more superoxide dismutases as disclosed herein. In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

The probiotic composition can comprise one or more soluble fiber as disclosed herein. In embodiments, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments, the probiotic of the probiotic composition comprises one or more beneficial microorganisms. In embodiments, the probiotic comprises bacteria of the genus Bifidobacterium. In embodiments, the probiotic comprises bacteria of the genus Lactobacillus. In embodiments, the probiotic comprises Firmicutes lactobacillus, Actinobacteria Bifidobacteriaceae or combinations thereof.

In embodiments, the probiotic composition is in the form of a gel. Examples of gels suitable for supplementing with a probiotic to make a probiotic composition are disclosed herein.

In embodiments, the probiotic composition is in the form of a powder. Examples of powders suitable for supplementing with a probiotic to make a probiotic composition are disclosed herein.

In embodiments, the probiotic composition is in the form of a liquid. Examples of liquids suitable for supplementing with a probiotic to make a probiotic composition are disclosed herein.

### 7) Excipients Formulations

In embodiments, the compositions disclosed herein can be combined with one or more excipient. In embodiments, the excipient is a gelling agent, a thickening agent, a carrier, a buffer or a filler. In embodiments, the compositions disclosed herein can be formulated into beverages or foodstuffs. In embodiments, the compositions are formulated into juice, a hydration beverage (e.g. a sports drink), a protein shake, tea, coffee, milk, kefir, ice cream, yogurt, a smoothie, broth or soup.

In embodiments, the compositions comprise the thickener/gelling agent carboxymethyl cellulose. In embodiments, composition is a gel comprising superoxide dismutase, soluble fiber and carboxymethyl cellulose. In embodiments, the composition comprises from about 0.5 to about 5.0 mg/mL carboxymethyl cellulose.

In embodiments, the compositions comprise the thickener/gelling agent xanthan gum. In embodiments, composition is a gel comprising superoxide dismutase, soluble fiber and xanthan gum. In embodiments, the composition comprises from about 0.5 to about 5.0 mg/mL xanthan gum.

### 8) Methods

The present disclosure is also directed to a method of increasing T-cell activation in a subject comprising orally administering to the subject a composition comprising: a) from about 10 units to about 200 units superoxide dismutase; and b) from about 500 mg to about 8000 mg soluble fiber; wherein, following administration of the composition, the activation of T cells is increased in the subject.

In embodiments, the activation of T cells is measured by measuring a biomarker of T cell activation as disclosed in the Examples below.

In embodiments, the composition is administered in combination with an anti-cancer agent. In embodiments, the composition is administered in combination with an anti-viral agent.

In embodiments of the method of increasing T cell activation, the method is used for preventing or treating viral infections, including infections of influenza A, influenza B, influenza C, influenza D, coronaviruses including SARS (severe acute respiratory syndrome), SARS-CoV-2 (causing COVID-19), MERS (Middle East respiratory syndrome), HIV, Ebola, rhinovirus and respiratory syncytial virus.

The present disclosure also provides a method of increasing the production of short chain fatty acids (SCFAs) in the digestive tract of a subject comprising orally administering to the subject a composition comprising: a) from about 10 units to about 200 units superoxide dismutase; and b) from about 500 mg to about 8000 mg soluble fiber; wherein, following administration of the composition, the production of SCFAs is increased in the digestive tract of the subject.

In embodiments, the SCFAs increased in production are acetate, propionate, butyrate, or lactate SCFAs, or combinations thereof. In embodiments, the SCFAs are increased in a manner that provides about the same ratio of acetate, propionate, butyrate, and lactate SCFAs compared to the ratio of acetate, propionate, butyrate, and lactate SCFAs prior to the increase.

The present disclosure also provides a method of increasing the amount of bacteria of the genus Bifidobacterium or Lactobacillus in the digestive tract of a subject comprising orally administering to the subject a composition comprising: a) from about 10 units to about 200 units superoxide dismutase; and b) from about 500 mg to about 8000 mg soluble fiber; wherein, following administration of the composition, the amount of bacteria of the genus Bifidobacterium, Lactobacillus, or combinations thereof, is increased in the digestive tract of the subject.

In embodiments of the method, the bacteria of the genus Bifidobacterium comprise the species Actinobacteria Bifidobacteriaceae. In embodiments, the bacteria of the genus Lactobacillus comprise the species Firmicutes lactobacillus.

In embodiments of the above methods, the composition used comprises from about 10 units to about 200 units superoxide dismutase. In embodiments, the composition comprises comprising from about 50 units to about 150 units superoxide dismutase. In embodiments, the composition comprises from about 70 units to about 100 units superoxide dismutase. In embodiments, the composition comprises from about 20 units to about 190 units superoxide dismutase. In embodiments, the composition comprises from about 30 units to about 180 units superoxide dismutase. In embodiments, the composition comprises from about 40 units to about 170 units superoxide dismutase. In embodiments, the composition comprises from about 50 units to about 160 units superoxide dismutase. In embodiments, the composition comprises from about 60 units to about 150 units superoxide dismutase. In embodiments, the composition comprises from about 70 units to about 140 units superoxide dismutase. In embodiments, the composition comprises from about 80 units to about 130 units superoxide dismutase. In embodiments, the composition comprises from about 90 units to about 130 units superoxide dismutase. In embodiments, the composition comprises from about 75 units to about 95 units superoxide dismutase. In embodiments, the composition comprises from about 80 units to about 90 units superoxide dismutase. In embodiments, the composition comprises about 20, 30, 40, 50, 60, 70, 80, 90, 100, 110, 120, 130, 140, 150, or 160 units superoxide dismutase. In embodiments, the composition comprises about 84 units superoxide dismutase.

In embodiments of the above methods, the composition used comprises from about 1000 mg to about 5000 mg soluble fiber. In embodiments, the composition comprises from about 2000 mg to about 4000 mg soluble fiber. In embodiments, the composition comprises from about 1000 mg to about 10000 mg soluble fiber. In embodiments, the composition comprises from about 2000 mg to about 9000 mg soluble fiber. In embodiments, the composition comprises from about 3000 mg to about 8000 mg soluble fiber. In embodiments, the composition comprises from about 2000 mg to about 4000 mg soluble fiber. In embodiments, the composition comprises about 500, 1000, 1500, 2000, 2500, 3000, 3500, 4000 4500 or 5000 mg soluble fiber.

In embodiments of the above methods, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

In embodiments of the above methods, the composition can comprise one or more superoxide dismutases as disclosed herein. In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the above methods, the composition can comprise one or more soluble fiber as disclosed herein. In embodiments, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments of the above methods, the composition is in the form of a gel. Examples of gels suitable for use in the above methods are disclosed herein.

In embodiments of the above methods, the composition is in the form of a powder. Examples of powders suitable for use in the above methods are disclosed herein.

In embodiments of the above methods, the composition is in the form of a liquid. Examples of liquids suitable for use in the above methods are disclosed herein.

In embodiments of any of the methods disclosed herein, the composition is administered to the mammal once a day. In embodiments of any of the methods disclosed herein, the composition is administered to the mammal twice a day. In embodiments of any of the methods disclosed herein, the composition is administered to the mammal three, four, five, six, seven, eight nine, ten times or more a day.

In embodiments of any of the methods disclosed herein, the mammal is a human. In embodiments, of any of the methods disclosed herein the mammal is a primate (e.g., monkey, ape, gorilla, macaque), a household animal (e.g., dog, cat, rabbit, hamster, Guinea pig, mouse, rat) or an agricultural animal (e.g., cow, sheep, horse, goat, pig).

### EXAMPLES

### Example 1: SOD and Soluble Fiber Gel Stimulates T-Cell Activation, Antioxidative and Anti-Inflammatory Pathway as Shown in vitro in Jurkat Cells

Background and Objective: REVIVIFY® pro-vitality antioxidant gel is composed of primary antioxidant superoxide dismutase (SOD), prebiotic fibers, diverse polyphenols from various fruits juice. REVIVIFY® has a formulation as shown in Table 4.

TABLE 4

| SI. # | Ingredient | QTY in mg |
|---|---|---|
| 1 | Soluble com fiber (Fibersol-2) | 4000 |
| 2 | Pomegranate Juice Concentrate | 4000 |
| 3 | Concord Grape Juice Concentrate | 4000 |
| 4 | Blueberry Juice Concentrate | 2000 |
| 5 | Dark Sweet Cherry Juice Concentrate | 1450 |
| 6 | Sour Cherry Juice Concentrate | 1450 |
| 7 | Goji Pulp | 294 |
| 8 | Acai Concentrate Juice | 200 |
| 9 | Aloe vera juice | 200 |
| 10 | Green Tea | 200 |
| 11 | Resveratrol | 80 |
| 12 | Extramel SOD (14000 unit/g) | 6 |
| 13 | CMC | 60.5 |
| 14 | Xanthan | 60.5 |
| 15 | QS Water (30 ml) | |

SOD diminishes the superoxide anion that is produced due to normal cellular activity. Polyphenols are phenolic compounds act as antioxidant, anti-inflammatory, and anti-viral agents. They repair damaged cells due to reactive oxygen molecules of ROS/RNS. Dietary prebiotic fibers modulate beneficiary gut eco microbiomes and provide many health benefits including increased immunity. The combination of these three components stimulates the immune system via T-cell activation and antioxidative and anti-inflammatory pathways. The objective of this study was to evaluate the effect of REVIVIFY® gel on an in vitro T cell Model.

Methods: The Jurkat Cell Line is an immortalized T lymphocyte cell line that has most often been used as a prototypical T cell line to study multiple events in T cell biology, including T cell signaling. Jurkat cells were seeded on 6 well plates. Prior to treatment, cells were incubated in serum free media for 24 hours. Cells were treated for 48 hours with the following agents: 1. Superoxide Dismutase only; 2. Prebiotic fiber only; 3. Fruit juice only; 4. superoxide Dismutase+Prebiotic fiber+Fruit juice (Combination); 5. Positive Control: Phorbol 12-myristate 13-acetate (PMA) in combination with ionomycin; 6. Negative Control: Cell culture media.

After treatment, media were removed from cells and placed in tubes. Levels of CD-8+; CD-4+; interferon-gamma (IFNγ); Interleukin-6 (IL-6); Interferon gamma-induced protein 10 (IP-10; also known as CXCL10); Macrophage inflammatory protein 1α and 1β; Monocyte chemoattractant protein 1 (MCP-1, also known as CCL2); and 8 isoprostane were measured by commercially available ELISA Kits.

Activated Jurkat Cells were seen by upregulated CD69 (measured using anti-CD69 antibody MCA2806A647, BioRad) expression on the CD3 (measured using anti-CD3 antibody MCA463A488) positive population of cells. Cells were gated on lymphocytes in the presence of Human Seroblock (BUF070A, BioRad). The Jurkat Cells were stimulated for five days with treatment and were stained with CytoTrack Red 628/643 by Cell Proliferation Assay Kit (1351205, BioRad). Data were acquired on the ZE5 Cell Analyzer. Data were expressed as mean±SE. Statistical significance were assessed by ANOVA and Duncan's post-hoc test for differences between treatment groups and treatment with negative control effects with p<0.05 was taken as significant. Results was presented as the mean±S.E. (n=6, four replicates).

Results: As discussed in detail below, REVIVIFY® and its components activated T cells are seen by upregulated CD69 expression and activated the differentiation of CD4+ and CD8+ compared to culture media. The gel and its components attenuated the Lipopolysaccharide-Induced Activation of 8-Isoprostane (81P), COX-2, IFN-γ, IL-6, TGF-β, TNF-α and CXCL10 Secretion by Jurkat Cells.

Conclusions/Perspectives: REVIVIFY® gel contains superoxide dismutase, prebiotic fiber and polyphenols and quercetin from Fruit juice. This unique multi direction approach to prevent oxidative stress, maintain pro-inflammatory and anti-inflammatory balance, and stimulate immune responses is very prompt and effective.

### Introduction

Superoxide dismutases (SODs): SODs constitute a very important antioxidant defense against oxidative stress in the body. The enzyme acts as a good therapeutic agent against reactive oxygen species-mediated diseases. SOD can have therapeutic effects in various physiological and pathological conditions such as cancer, inflammatory diseases, cystic fibrosis, ischemia, aging, rheumatoid arthritis, neurodegenerative diseases, and diabetes. However, the enzyme has certain limitations in clinical applications due to issues with absorption. Therefore, SOD conjugates and mimetics have been developed to increase its therapeutic efficiency^{1,2,3,4,5,6,7}.

Polyphenols: Polyphenols are secondary metabolites of plants and are generally involved in defense against ultraviolet radiation or aggression by pathogens. In the last decade, there has been much interest in the potential health benefits of dietary plant polyphenols as antioxidant. Epidemiological studies and associated meta-analyses strongly suggest that long term consumption of diets rich in plant polyphenols offer protection against development of cancers, cardiovascular diseases, diabetes, osteoporosis and neurodegenerative diseasess,^{9,10,11,12}.

Dietary prebiotic fibers: The health benefits of dietary fiber have long been appreciated. Higher intakes of dietary fiber are linked to less cardiovascular disease and fiber plays a role in gut health, with many effective laxatives actually isolated fiber sources. Higher intakes of fiber are linked to lower body weights. Only polysaccharides were included in dietary fiber originally, but more recent definitions have included oligosaccharides as dietary fiber, not based on their chemical measurement as dietary fiber by the accepted total dietary fiber (TDF) method, but on their physiological effects. Inulin, fructo-oligosaccharides, and other oligosaccharides are included as fiber in food labels in the US. Additionally, oligosaccharides are the best known “prebiotics,” “a selectively fermented ingredient that allows specific changes, both in the composition and/or activity in the gastrointestinal microflora that confers benefits upon host well-bring and health.” To date, all known and suspected prebiotics are carbohydrate compounds, primarily oligosaccharides, known to resist digestion in the human small intestine and reach the colon where they are fermented by the gut microflora. Studies have provided evidence that inulin and oligofructose (OF), lactulose, and resistant starch (RS) meet all aspects of the definition, including the stimulation of Bifidobacterium, a beneficial bacterial genus. Other isolated carbohydrates and carbohydrate-containing foods, including galactooligosaccharides (GOS), transgalactooligosaccharides (TOS), polydextrose, wheat dextrin, acacia gum, psyllium, banana, whole grain wheat, and whole grain corn also have prebiotic effects^{13,14,15,16,17}.

T cell activation and immunity: T cells are generated in the Thymus and are programmed to be specific for one particular foreign particle (antigen). Once they leave the thymus, they circulate throughout the body until they recognize their antigen on the surface of antigen presenting cells (APCs). The T cell receptor (TCR) on both CD4⁺ helper T cells and CD8⁺ cytotoxic T cells binds to the antigen as it is held in a structure called the MHC complex, on the surface of the APC. This triggers initial activation of the T cells. The CD4 and CD8 molecules then bind to the MHC molecule too, stabilizing the whole structure. This initial binding between a T cell specific for one antigen and the antigen-MHC it matches sets the whole response in motion. This normally takes place in the secondary lymphoid organs¹⁸.

Role of T cells in COVID-19 infection: Like B cells, which produce antibodies, T cells are central players in the immune response to viral infection¹⁹. When the SARS-CoV-2 virus, which causes COVID-19, infects epithelial cells, such as those found in the airways, it replicates inside the cells, using the host cell's biochemical machinery. This causes the host cell to undergo programmed cell death, releasing molecules called damage-associated molecular patterns (e.g. nucleic acids and oligomers)²⁰. These molecules are recognized by macrophages and neighboring endothelial and epithelial cells, causing them to produce pro-inflammatory cytokines, including chemokines: Interleukin-6 (IL-6); Interferon gamma-induced protein 10 (IP-10; also known as CXCL10); Macrophage inflammatory protein 1α and 1β; Monocyte chemoattractant protein 1 (MCP-1, also known as CCL2). Monocytes, macrophages, and T cells are then recruited to the site of infection by these chemokines and other cytokines and promote further inflammation. As part of this inflammatory response, the recruited T cells produce interferon-gamma (IFNγ).

Several types of T cells are involved in this response. CD4+ T helper (Th) cells interact with CD8+ T cells, which drive the cytotoxic response that kills cells infected with the virus. The CD8+ T cells directly recognize viral peptides presented at the surfaces of infected cells, causing apoptosis (a form of programmed cell death) and preventing the virus from spreading further. Follicular helper T (TF_{H}) cells are a specialized subset of CD4+ T cells that provide help to B cells through both cell-cell interactions and release of cytokines, leading to the production of antibodies by B cells¹⁹. These neutralizing antibodies can recognize whole viruses and act by blocking the virus from infecting cells. Alveolar macrophages recognize the neutralized viruses and the apoptotic cells (killed by the CD8+ T cells) and clear them by phagocytosis. This then results in recovery from the viral infection²⁰.

Studies assessing the clinical features of patients infected with SARS-CoV-2 have reported an incubation time of 4 to 7 days before the onset of symptoms, and a further 7 to 10 days before progression to severe disease²¹.

For many primary virus infections, it typically takes 7 to 10 days to prime and expand adaptive T cell immune responses to control the virus, and this correlates with the typical time it takes for patients with COVID-19 either to recover or to develop severe illness²². This raises the possibility that a poor initial T cell response contributes to persistence and severity of SARS-CoV-2, whereas early strong T cell responses may be protective.

The CD4+ T cell response in COVID-19: Some studies have shown that in patients with severe COVID-19 there is evidence of impaired function of CD4+ T cells, including reduced IFNγ production²², while others seem to suggest over-activation of these T cells²³.

Overall, the CD4+ T cell response in acute SARS-CoV-2 infection, whether impaired, over-activated, or inappropriate, and how this relates to disease outcomes, remains to be elucidated and is an important question. A particularly high frequency of CD4+ T cell responses specific to virus spike protein has been observed in patients who have recovered from COVID-19, which is similar to what has been reported for influenza virus infections²¹. In one small study of 14 patients, circulating virus-specific CD4+ T cells were identified in all of those who recovered from SARS-CoV-2, which also suggests the potential for developing T cell memory²⁴ and perhaps longer-term immunity.

The CD8+ T cell response in COVID-19: There appears to be heterogeneity in the immune response between patients. Some studies have reported that CD8+ T cells from patients with severe COVID-19 had reduced cytokine production following in vitro stimulation, and some have shown evidence of possibly exhausted T cells; in contrast, other studies have reported an overaggressive CD8+ T cell response or highly activated CD8+ T cells with increased cytotoxic response in patients with COVID-19²⁵.

### Experimental Procedure Research Strategy

A model cell system is used to test this hypothesis. The Jurkat cell line is an immortalized T lymphocyte cell line that was originally obtained from the peripheral blood of a boy with T cell leukemia²⁶. The Jurkat cell line has most often been used as a prototypical T cell line to study multiple events in T cell biology, including a) T cell signaling and b) molecular events in the HIV infection life cycle. In T cell signaling¹⁸, the Jurkat cell line has been used to model and characterize signaling events in T cell activation (TCA), a critical process in effective adaptive immune response²⁶. As a model signaling axis, TCA involves surface signaling through the T cell receptor (TCR) and accessory proteins CD3 and CD28 on the surface of T cells and initiates a cascade of molecular events that result in transcriptional activation of multiple genes, including the interleukin-2 (IL-2) gene, a canonical T cell activation target gene. Steps in TCA include the activation of a series of kinases (e.g. LCK, JNK, PKC) and phosphatase proteins (Calcineurin), as well as activation of quiescent cytoplasmic transcription factors (e.g. NF-kB, NFAT), which, upon activation, translocate into the nucleus to activate target genes. Each of these steps, and many other intermediate factors in this pathway, has been dissected using mutant subclones of the Jurkat T cell line, which has been instrumental in mapping the signaling pathways and identifying critical players that underlie T cell activation^{18,26}.

Jurkat Cell Line Culture (Jurkat, Clone E6-1 (ATCC® TIB-152™; Human; Homo sapiens)

Complete Growth Medium: The base medium for this cell line is ATCC-formulated RPMI-1640 Medium, ATCC 30-2001. To make the complete growth medium, the following component is added to the base medium: fetal bovine serum (ATCC 30-2020) to a final concentration of 10%.

Subculturing: Cultures are maintained by the addition of fresh medium or replacement of medium. Alternatively, cultures are established by centrifugation with subsequent resuspension at 1×10⁵ viable cells/mL. Cell density should not be allowed to exceed 3×10⁶ cells/mL. Corning® T-75 flasks are recommended for subculturing this product.

Interval: Cultures were maintained at a cell concentration between 1×10⁵ and 1×10⁶ viable cells/mL.

Medium Renewal: Fresh medium was added every 2 to 3 days (depending on cell density).

Culture Conditions: atmosphere: air, 95%; carbon dioxide (CO₂), 5%; temperature: 37° C.

Treatment of Jurkat Cells With A Dietary Supplement of Superoxide Dismutase in Combination with Prebiotic Fiber Containing Polyphenols from Fruit Juices

Effect of superoxide dismutase in combination with prebiotic fiber containing polyphenols from fruit juices on Jurkat Cells: Jurkat cells were seeded on 6 well plates. Prior to treatment, cells were incubated in serum free media for 24 hours. Cells were treated for 48 hours with the following agents: 1. Superoxide Dismutase only; 2. Prebiotic fiber only; 3. Fruit juice only; 4. superoxide Dismutase+Prebiotic fiber+Fruit juice (Combination); 5. Positive Control: Phorbol 12-myristate 13-acetate (PMA) in combination with ionomycin; 6. Negative Control: Cell culture media.

Enzyme-Linked Immunosorbent Assay: After treatment, the media was removed from cells were placed in tubes. To evaluate the T-cell activation the following factors were measured in the cell medium. Levels of CD-8+; CD-4+; interferon-gamma (IFNγ); Interleukin-6 (IL-6); Interferon gamma-induced protein 10 (IP-10; also known as CXCL10); Macrophage inflammatory protein 1α and 1β; Monocyte chemoattractant protein 1 (MCP-1, also known as CCL2); and 8 isoprostane by commercially available ELISA Kits as described previously^{27,28,29,30,31,32,33,34}.

T cell stimulation for flow cytometry analysis: Cells were treated with following agents for 48 hours: 1. Superoxide Dismutase only; 2. Prebiotic fiber only; 3. Fruit juice only; 4. superoxide Dismutase+Prebiotic fiber+Fruit juice (Combination); 5. Positive Control: Phorbol 12-myristate 13-acetate (PMA) in combination with ionomycin; 6. Negative Control: Cell culture media.

Activated Jurkat cells were detected as described above and statistical analysis of the results was performed as described above.

### Results

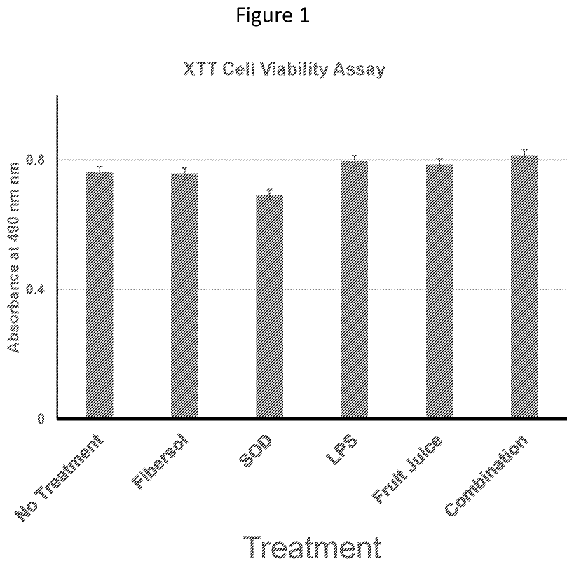

Cell viability assay: The assay determines the ability of living cells to convert a redox dye, resazurin into a fluorescent end product, resorufin. Jurkat cells were seeded onto 96-well plates in complete medium and allowed to adhere overnight at 37° C. Cells were then treated with vehicle (Cell culture media) or Prebiotic fiber only, Superoxide Dismutase only, Fruit juice only, Superoxide Dismutase+Prebiotic fiber+Fruit juice (Combination) or LPS (positive control). After incubation for 48 h with the respective treatments, 20 μl of Cell Titer-Blue reagent was added to each well. Absorbance at 520 nm was determined by a microtiter plate reader. The signal produced by the conversion of resazurin to resorufin is directly proportional to the number of viable cells. As shown in FIG. 1, none of the gel components or Combination showed toxic effects in cell viability assays.

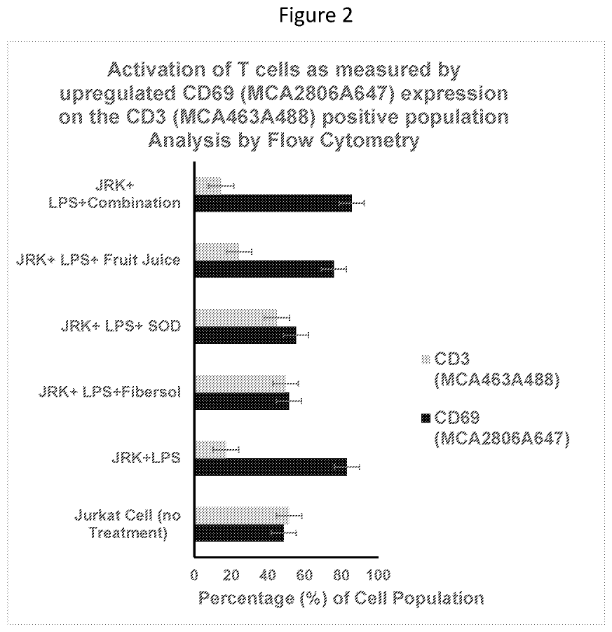

The gel and its components activate T cells as seen by upregulated CD69 expression on the CD3 positive cell population with analysis by Flow Cytometry: T cell stimulation for flow cytometry analysis. Activation and proliferation protocols provide an effective method to determine immunocompetence and cell reactivity. Jurkat cells were stimulated with vehicle (cell culture media) or prebiotic fiber only, superoxide dismutase only, fruit juice only, superoxide dismutase+prebiotic fiber+fruit juice (Combination) or LPS (positive control). Cells were gated on lymphocytes in the presence of Human Seroblock. Data were acquired on the ZE5™ Cell Analyzer. Activated T cells are seen by upregulated CD69 expression on the CD3 positive population as shown in FIG. 2. As shown in FIG. 2, the Combination showed a surprisingly high level of T cell activation as measured by CD69 expression.

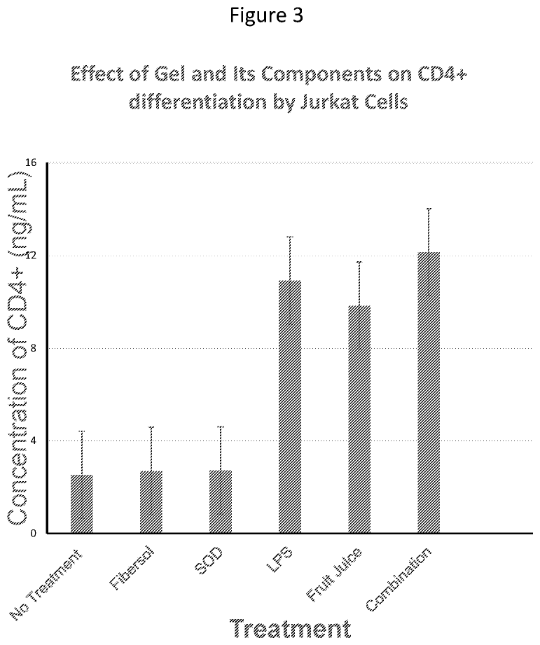

Effect of Original Components of REVIVIFY® on CD4⁺ differentiation by Jurkat Cells. Jurkat cells were stimulated with vehicle (Cell culture media) or Prebiotic fiber only, Superoxide Dismutase only, Fruit juice only, Superoxide Dismutase+Prebiotic fiber+Fruit juice (Combination). The concentration of CD4⁺ (ng/mL) was measured by ELISA according to the manufacturer's protocol. As shown in FIG. 3, Fruit juice and Superoxide Dismutase+Prebiotic fiber+Fruit juice (Combination) significantly and surprisingly activated the differentiation of CD4+ compared to culture media as determined by ELISA.

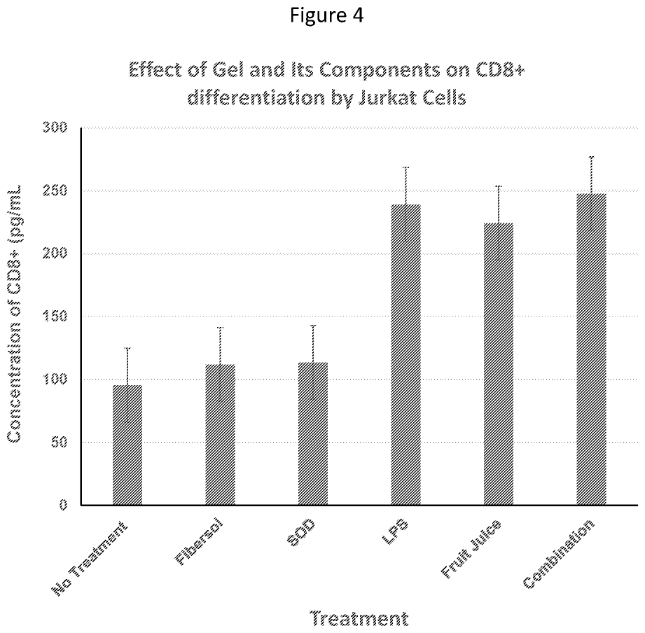

Effect of Original Components of REVIVIFY® on CD8⁺ differentiation by Jurkat Cells. Jurkat cells were stimulated with vehicle (Cell culture media) or Prebiotic fiber only, Superoxide Dismutase only, Fruit juice only, Superoxide Dismutase+Prebiotic fiber+Fruit juice (Combination). The concentration of CD8⁺ (pg/mL) was measured by ELISA according to the manufacturer's protocol. As shown in FIG. 4, Fruit juice and Superoxide Dismutase+Prebiotic fiber+Fruit juice (Combination) significantly and surprisingly activated the differentiation of CD4+ compared to culture media as determined by ELISA.

The concentration ratio of CD4⁺ and CD8⁺ in REVIVIFY® treated Jurkat cells: The concentration ratio of CD4⁺/CD8⁺ in the REVIVIFY® treated Jukart cells is 48:1 that is higher than the peripheral blood of healthy adults and mice³⁵. The CD4⁺/CD8⁺ ratio is the ratio of T helper cells (with the surface marker CD4) to cytotoxic T cells (with the surface marker CD8). Both CD4⁺ and CD8⁺ T cells contain several subsets.³⁶ The CD4⁺/CD8⁺ ratio in the peripheral blood of healthy adults and mice is about 2:1, and an altered ratio can indicate diseases relating to immunodeficiency³⁵. This large difference in the ratio is due to the experiment in in vitro closed system. In the other hand, it also indicates the high potential of REVIVIFY® to stimulate T-cell for higher differentiation into CD4⁺. These data indicate that follicular helper T (TFH) cells are a specialized subset of CD4⁺ T cells that provide help to B cells through both cell-cell interactions and release of cytokines, leading to the production of antibodies by B cells¹⁹. These neutralizing antibodies can recognize whole viruses and act by blocking the virus from infecting cells. Alveolar macrophages recognize the neutralized viruses and the apoptotic cells (killed by the CD8+ T cells) and clear them by phagocytosis. This then results in recovery from the viral infection²⁰. In coronavirus disease 2019 (COVID-19) B cell, natural killer cell, and total lymphocyte counts decline, but both CD4+ and CD8+ cells decline to a far greater extent.³⁷ Low CD4+ predicted greater likelihood of intensive care unit admission, and CD4+ cell count was the only parameter that predicted length of time for viral RNA clearance.³⁷

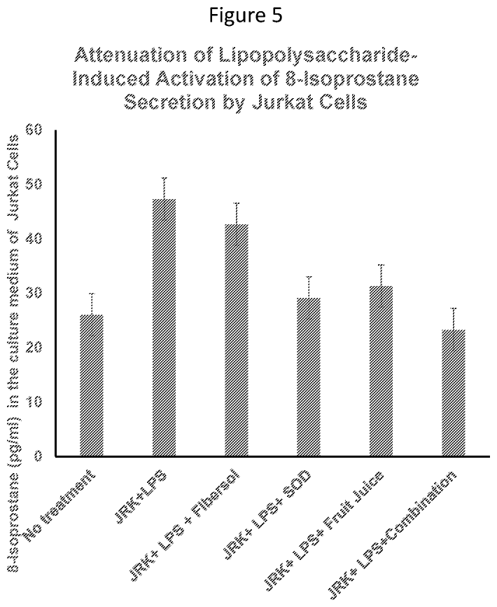

REVIVIFY® gel has antioxidant activity: As shown in FIG. 5, REVIVIFY® and its components attenuate the Lipopolysaccharide-Induced Activation of 8-Isoprostane Secretion by Jurkat Cells in the in vitro study. Lipopolysaccharide (LPS)-stimulated Jurkat Cells were treated with vehicle (Cell culture media) or Prebiotic fiber only, Superoxide Dismutase only, Fruit juice only, Superoxide Dismutase+Prebiotic fiber+Fruit juice (Combination). The levels of 8IP in the culture media of were measured by ELISA. REVIVIFY® and its components attenuated the Lipopolysaccharide-Induced Activation of 8-Isoprostane (8IP) Secretion by Jurkat Cells. The level of 8IP has been proposed as a marker of antioxidant deficiency and oxidative stress^{38,39}. As seen in FIG. 5, the Combination surprisingly reduced 8IP levels to below those seen in untreated cells.

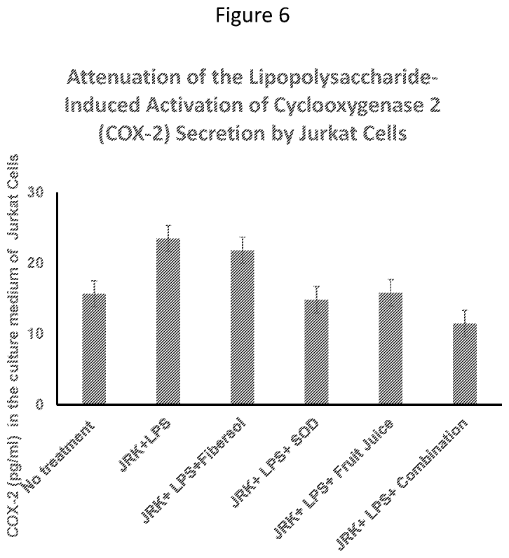

REVIVIFY® gel has anti hypoxia activity: As shown in FIG. 6, REVIVIFY® and its components attenuates the Lipopolysaccharide-Induced Activation of Cyclooxygenase-2 (COX-2) Secretion by Jurkat Cells in the in vitro study. Lipopolysaccharide (LPS)-stimulated Jurkat Cells were treated with vehicle (Cell culture media) or Prebiotic fiber only, Superoxide Dismutase only, Fruit juice only, Superoxide Dismutase+Prebiotic fiber+Fruit juice (Combination). Levels of COX-2 in the culture media were measured by ELISA. As seen in FIG. 6, the Combination surprisingly reduced COX-2 levels to below those seen in untreated cells.

REVIVIFY® and its components attenuated the Lipopolysaccharide-Induced Activation of COX-2 Secretion by Jurkat Cells (FIG. 6): Hypoxia increases COX-2 expression⁴⁰. Apigenin down-regulates COX-2 expression in lupus T cells, B cells and antigen-presenting cells, and causes their apoptosis⁴¹. Although no clear structural/functional relationships have been established, and without wishing to be bound by theory, it appears that the C-2,3-double bond and the hydroxyl substitutions on A- and B-rings are important contributors to this inhibitory activity⁴². Animal data confirm down-regulation of COX-2 expression in different inflammatory diseases⁴³.

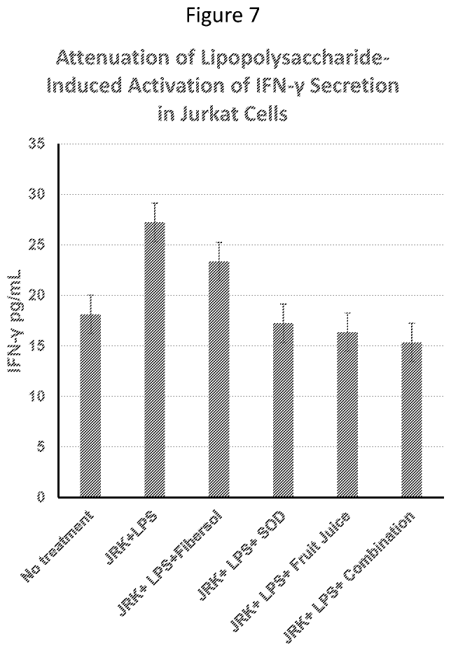

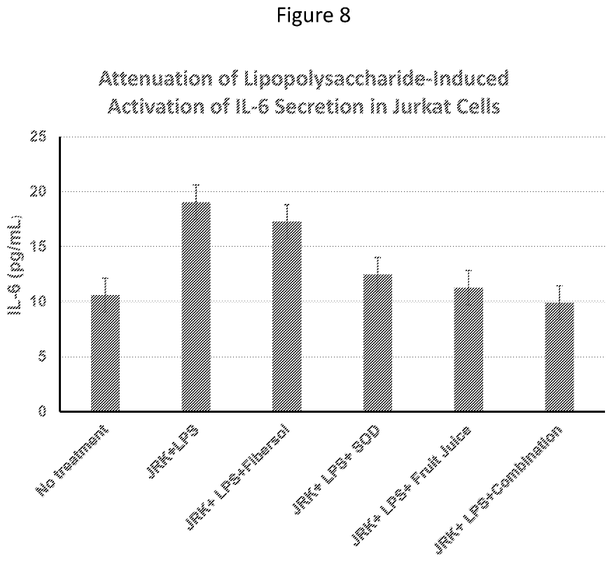

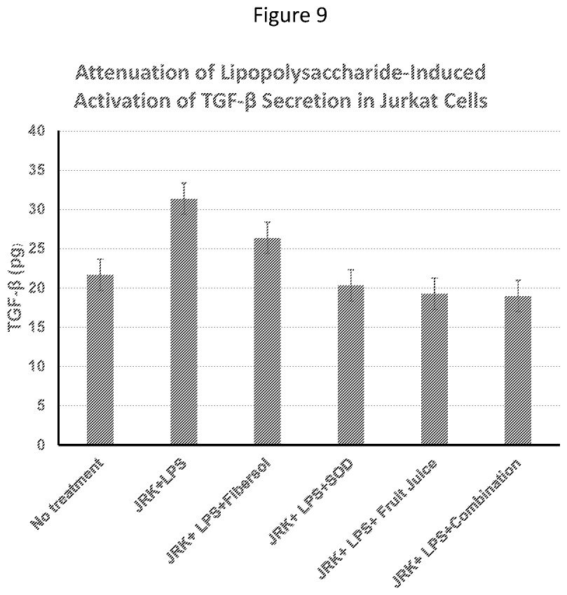

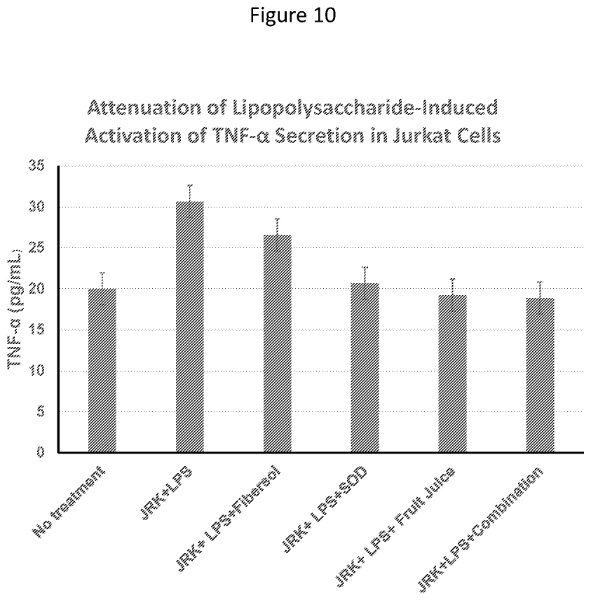

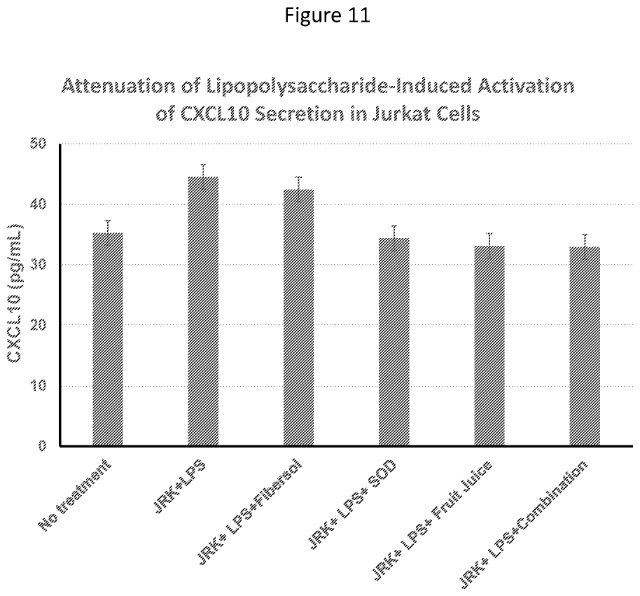

REVIVIFY® gel has anti-inflammatory activity: REVIVIFY® and its components attenuate the Lipopolysaccharide-Induced Activation of inflammatory activities in the in vitro Jurkat Cells study. Lipopolysaccharide (LPS)-stimulated Jurkat Cells were treated with vehicle (Cell culture media) or Prebiotic fiber only, Superoxide Dismutase only, Fruit juice only, Superoxide Dismutase+Prebiotic fiber+Fruit juice (Combination). The levels of Interferon gamma (IFNγ, FIG. 7), Interleukin 6 (IL-6, FIG. 8), Transforming growth factor beta (TGF-β, FIG. 9), Tumor necrosis factor (TNF, FIG. 10) and C-X-C motif chemokine ligand 10 (CXCL10, FIG. 11) in the culture media were measured by commercially available ELISA kits.

REVIVIFY® and its components attenuates the Lipopolysaccharide-Induced Activation of IFN-γ Secretion by Jurkat Cells (FIG. 7): Interferon gamma (IFNγ) is a dimerized soluble cytokine that is the only member of the type II class of interferons.⁴⁴The existence of this interferon, which early in its history was known as immune interferon, was described by E. F. Wheelock as a product of human leukocytes stimulated with phytohemagglutinin, and by others as a product of antigen-stimulated lymphocytes.⁴⁵ IFNγ, or type II interferon, is a cytokine that is critical for innate and adaptive immunity against viral, some bacterial and protozoan infections. IFNγ is an important activator of macrophages and inducer of major histocompatibility complex class II molecule expression. Aberrant IFNγ expression is associated with a number of autoinflammatory and autoimmune diseases. The importance of IFNγ in the immune system stems in part from its ability to inhibit viral replication directly, and most importantly from its immunostimulatory and immunomodulatory effects. IFNγ is produced predominantly by natural killer cells (NK) and natural killer T cells (NKT) as part of the innate immune response, and by CD4 Th1 and CD8 cytotoxic T lymphocyte (CTL) effector T cells once antigen-specific immunity develops^{46,47} as part of the adaptive immune response. IFNγ is also produced by non-cytotoxic innate lymphoid cells (ILC), a family of immune cells first discovered in the early 2010s.⁴⁸ As seen in FIG. 7, cells treated with SOD, fruit juice polyphenols and the Combination surprisingly all have lower levels of IFNγ secretion than untreated cells, with the lowest levels of secretion seen in cells treated by the Combination.

REVIVIFY® and its components attenuate the Lipopolysaccharide-Induced Activation of IL-6 Secretion by Jurkat Cells (in FIG. 8): Interleukin 6 (IL-6) is an interleukin that acts as both a pro-inflammatory cytokine and an anti-inflammatory myokine. In humans, it is encoded by the IL6 gene.⁴⁹ In addition, osteoblasts secrete IL-6 to stimulate osteoclast formation. Smooth muscle cells in the tunica media of many blood vessels also produce IL-6 as a pro-inflammatory cytokine. IL-6's role as an anti-inflammatory myokine is mediated through its inhibitory effects on TNF-alpha and IL-1 and its activation of IL-1ra and IL-10. There is some early evidence that IL-6 can be used as an inflammatory marker for severe COVID-19 infection with poor prognosis, in the context of the wider coronavirus pandemic.⁵⁰ IL-6 is secreted by macrophages in response to specific microbial molecules, referred to as pathogen-associated molecular patterns (PAMPs). These PAMPs bind to an important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. IL-6 is an important mediator of fever and of the acute phase response. IL-6 is responsible for stimulating acute phase protein synthesis, as well as the production of neutrophils in the bone marrow. It supports the growth of B cells and is antagonistic to regulatory T cells. As seen in FIG. 8, the Combination surprisingly reduced IL-6 activation levels to below the levels seen in untreated cells.

REVIVIFY® and its components attenuate the Lipopolysaccharide-Induced Activation of TGF-β Secretion by Jurkat Cells (in FIG. 9): Transforming growth factor beta (TGF-β) is a multifunctional cytokine belonging to the transforming growth factor superfamily that includes three⁵¹. TGFB proteins are produced by all white blood cell lineages. Activated TGF-β complexes with other factors to form a serine/threonine kinase complex that binds to TGF-β receptors. TGF-β receptors are composed of both type 1 and type 2 receptor subunits. After the binding of TGF-β, the type 2 receptor kinase phosphorylates and activates the type 1 receptor kinase that activates a signaling cascade.⁵² This leads to the activation of different downstream substrates and regulatory proteins, inducing transcription of different target genes that function in differentiation, chemotaxis, proliferation, and activation of many immune cells.^{52,53}

TGF-β is secreted by many cell types, including macrophages, in a latent form in which it is complexed with two other polypeptides, latent TGF-beta binding protein (LTBP) and latency-associated peptide (LAP). Serum proteinases such as plasmin catalyze the release of active TGF-β from the complex. This often occurs on the surface of macrophages where the latent TGF-β complex is bound to CD36 via its ligand, thrombospondin-1 (TSP-1). Inflammatory stimuli that activate macrophages enhance the release of active TGF-β by promoting the activation of plasmin. Macrophages can also endocytose IgG-bound latent TGF-β complexes that are secreted by plasma cells and then release active TGF-β into the extracellular fluid.⁵⁴ Among its key functions is regulation of inflammatory processes, particularly in the gut.[5] TGF-β also plays a crucial role in stem cell differentiation as well as T-cell regulation and differentiation.^{56,57} Because of its role in immune and stem cell regulation and differentiation, it is a highly researched cytokine in the fields of cancer, auto-immune diseases, and infectious disease.

The TGF-β superfamily includes endogenous growth inhibiting proteins; an increase in expression of TGF-β often correlates with the malignancy of many cancers and a defect in the cellular growth inhibition response to TGF-β. Its immunosuppressive functions then come to dominate, contributing to oncogenesis.⁵⁸ The dysregulation of its immunosuppressive functions is also implicated in the pathogenesis of autoimmune diseases, although their effect is mediated by the environment of other cytokines present.” TGF-β induces apoptosis, or programmed cell death, in human lymphocytes and hepatocytes. The importance of this function is clear in TGF-β deficient mice which experience hyperproliferation and unregulated autoimmunity.⁵⁹ As seen in FIG. 9, cells treated with SOD, fruit juice polyphenols and the Combination surprisingly all have lower levels of TGF-β secretion than untreated cells, with the lowest levels of secretion seen in cells treated by the Combination.

REVIVIFY® and its components attenuates the Lipopolysaccharide-Induced Activation of TNF-α Secretion by Jurkat Cells (in FIG. 10): Tumor necrosis factor (TNF, cachexin, or cachectin; often called tumor necrosis factor alpha or TNF-α) is a cytokine—a small protein used by the immune system for cell signaling. If macrophages (certain white blood cells) detect an infection, they release TNF to alert other immune system cells as part of an inflammatory response. TNF is a member of the TNF superfamily, which consists of various transmembrane proteins with a homologous TNF domain. TNF was thought to be produced primarily by macrophages,⁶⁰ but it is produced also by a broad variety of cell types including lymphoid cells, mast cells, endothelial cells, cardiac myocytes, adipose tissue, fibroblasts, and neurons.⁶¹ Large amounts of TNF are released in response to lipopolysaccharide, other bacterial products, and interleukin-1 (IL-1). In the skin, mast cells appear to be the predominant source of pre-formed TNF, which can be released upon inflammatory stimulus (e.g., LPS).⁶² It has a number of actions on various organ systems, generally together with IL-1 and interleukin-6 (IL-6). A local increase in concentration of TNF will cause the cardinal signs of Inflammation to occur: heat, swelling, redness, pain and loss of function. Whereas high concentrations of TNF induce shock-like symptoms, the prolonged exposure to low concentrations of TNF can result in cachexia, a wasting syndrome. This can be found, for example, in cancer patients⁶³.

Said et al. showed that TNF causes an IL-10-dependent inhibition of CD4 T-cell expansion and function by up-regulating PD-1 levels on monocytes which leads to IL-10 production by monocytes after binding of PD-1 by PD-L.⁶⁴ The research of Pedersen et al. indicates that TNF increase in response to sepsis is inhibited by the exercise-induced production of myokines. To study whether acute exercise induces a true anti-inflammatory response, a model of ‘low grade inflammation’ was established in which a low dose of E. coli endotoxin was administered to healthy volunteers, who had been randomized to either rest or exercise prior to endotoxin administration. In resting subjects, endotoxin induced a 2- to 3-fold increase in circulating levels of TNF. In contrast, when the subjects performed 3 hours of ergometer cycling and received the endotoxin bolus at 2.5 h, the TNF response was totally blunted.⁶⁵ This study provides some evidence that acute exercise may inhibit TNF production.⁶⁶ In the brain TNF can protect against excitotoxicity.⁶⁷ TNF strengthens synapses.⁶⁸ TNF in neurons promotes their survival, whereas TNF in macrophages and microglia results in neurotoxins that induce apoptosis.⁶⁷ As seen in FIG. 10, cells treated with fruit juice polyphenols and the Combination surprisingly both have lower levels of TNF-alpha secretion than untreated cells, with the lowest levels of secretion seen in cells treated by the Combination.

REVIVIFY® and its components attenuate the Lipopolysaccharide-Induced Activation of CXCL10 Secretion by Jurkat Cells (in FIG. 11): C-X-C motif chemokine ligand 10 (CXCL10) also known as Interferon gamma-induced protein 10 (IP-10) or small-inducible cytokine B10 is an 8.7 kDa protein that in humans is encoded by the CXCL10 gene.^{69,70} C-X-C motif chemokine 10 is a small cytokine belonging to the CXC chemokine family. CXCL10 is secreted by several cell types in response to IFN-γ. These cell types include monocytes, endothelial cells and fibroblasts.⁶⁹ CXCL10 has been attributed to several roles, such as chemoattraction for monocytes/macrophages, T cells, NK cells, and dendritic cells, promotion of T cell adhesion to endothelial cells, antitumor activity, and inhibition of bone marrow colony formation and angiogenesis.^{71,72} This chemokine elicits its effects by binding to the cell surface chemokine receptor CXCR3.⁷³ As seen in FIG. 10, cells treated with SOD, fruit juice polyphenols and the Combination surprisingly all have lower levels of CXCL10 secretion than untreated cells, with the lowest levels of secretion seen in cells treated by the Combination.

A summary of all of the ELISA results discussed above is provided in Table 5.

TABLE 5
Effects (means with SE) of treatments on JRK cells with 6 replicates per treatment

| | Treatment of JRK cells using polysaccharide with additives | | | | | | | | | | |
|---|---|---|---|---|---|---|---|---|---|---|---|
| | | | Fiberosol | | | | Fruit juice | | Finished | | |
| | No | | fiber (1.3 | | SOD (0.2 | | (4.6 | | Product (5.6 | | |
| Variable | treatment | | mg/mL) | | mg/mL) | | mg/mL) | | mg/mL) | | P-level |
| CXCL10 | 35.2 | (0.5)^{A} | 42.5 | (0.4)^{B} | 34.3 | (0.4)^{AC} | 33.2 | (0.5)^{CD} | 32.5 | (0.4)^{D} | <0.001# |
| (pg/mL) | | | | | | | | | | | |
| CD4+ (ng/mL) | 2.5 | (0.1)^{A} | 2.7 | (0.1)^{AC} | 2.7 | (0.1)^{AC} | 9.8 | (0.4)^{BC} | 12.1 | (0.3)^{B} | <0.001* |
| CD8+ (ng/mL) | 95.5 | (3.4)^{A} | 112.0 | (1.9)^{B} | 113.5 | (2.7)^{B} | 224.3 | (5.6)^{C} | 247.7 | (2.9)^{D} | <0.001# |
| XTT cell | 0.76 | (0.02)^{A} | 0.76 | (0.02)^{A} | 0.69 | (0.01)^{B} | 0.79 | (0.02)^{AC} | 0.82 | (0.01)^{C} | <0.001# |
| viability | | | | | | | | | | | |
| (absorbance at | | | | | | | | | | | |
| 490 nm) | | | | | | | | | | | |
| 8-isoprostane | 26.1 | (0.9)^{AD} | 42.7 | (1.7)^{B} | 29.2 | (1.4)^{AC} | 31.3 | (1.7)^{C} | 23.3 | (0.7)^{D} | <0.001# |
| (pg/mL) | | | | | | | | | | | |
| COX-2 (pg/mL) | 15.7 | (1.1)^{A} | 21.8 | (0.9)^{B} | 14.8 | (0.7)^{A} | 15.8 | (0.9)^{A} | 11.5 | (0.4)^{C} | <0.001# |
| INF-y (pg/mL) | 18.3 | (0.7)^{A} | 23.3 | (0.4)^{B} | 17.2 | (0.4)^{AC} | 16.2 | (0.3)^{CD} | 15.2 | (0.3)^{D} | <0.001# |
| IL-6 (pg/mL) | 10.3 | (0.3)^{A} | 17.2 | (0.7)^{B} | 12.2 | (0.3)^{C} | 11.3 | (0.4)^{AC} | 9.4 | (0.3)^{A} | <0.001# |
| TGF-beta | 21.7 | (0.7)^{A} | 26.2 | (0.7)^{B} | 20.2 | (0.5)^{AC} | 19.2 | (0.5)^{C} | 18.5 | (0.4)^{C} | <0.001# |
| (pg/mL) | | | | | | | | | | | |
| TNF-alpha | 19.5 | (0.6)^{AC} | 26.7 | (0.5)^{B} | 20.3 | (0.7)^{C} | 19.3 | (0.7)^{AC} | 18.3 | (0.5)A | <0.001# |
| (pg/mL) | | | | | | | | | | | |

### REFERENCES FOR EXAMPLE 1

1. Kangralkar V A, Patil S D, Bandivadekar R M. Oxidative stress and diabetes: A review. Intl J Pharm Appl. 2010; 1:38-45.
2. Yasui K, Baba A. Therapeutic potential of superoxide dismutase (SOD) for resolution of inflammation. Inflamm Res. 2006; 55:359-63.
3. Landis G N, Tower J. Superoxide dismutase evolution and life span regulation. Mech Ageing Dev. 2005; 126:365-79.
4. Noor R, Mittal S, Iqbal J. Superoxide dismutase—Applications and relevance to human diseases. Med Sci Monit. 2002; 8:RA210-5.
5. Inal M E, Kanbak G, Sunal E. Antioxidant enzyme activities and malondialdehyde levels related to aging. Clin Chim Acta. 2001; 305:75-80.
6. Riley D P. Functional mimics of superoxide dismutase enzymes as therapeutic agents. Chem Rev. 1999; 99:2573-88.
7. Salvemini D, Riley D P. Nonpeptidyl mimetics of superoxide dismutase in clinical therapies for diseases. Cell Mol Life Sci. 2000; 57:1489-92.
8. Scalbert A, Manach C, Morand C, Remesy C. Dietary polyphenols and the prevention of diseases. Crit Rev Food Sci Nutr. 2005; 45:287-306.
9. Beckman C H. Phenolic-storing cells: keys to programmed cell death and periderm formation in wilt disease resistance and in general defense responses in plants?Physiol. Mol. Plant Pathol. 2000; 57:101-110.
10. Arts I C W, Hollman P C H. Polyphenols and disease risk in epidemiologic studies. Am J Clin Nutr. 2005; 81:317-325.
11. Kondratyuk T P, Pezzuto J M. Natural Product Polyphenols of Relevance to Human Health. Pharm Biol. 2004; 42:46-63.
12. Shahidi F, Naczk M. Food phenolics, sources, chemistry, effects, applications. Lancaster, PA: Technomic Publishing Co Inc; 1995.
13. Slavin J. L. Dietary fiber: Classification, chemical analyses, and food sources. J. Am. Diet. Assoc. 1987; 87:1164-1171.
14. Gibson G. R., Roberfroid M. B. Dietary modulation of the human colonic microbiota: Introducing the concept of prebiotics. J. Nutr. 1995; 125:1401-1412.
15. Gibson G. R., Probert H. M., van Loo J., Rastall R. A., Roberfroid M. B. Dietary modulation of the human colonic microbiota: Updating the concept of prebiotics. Nutr. Res. Rev. 2004; 17:259-275. doi: 10.1079/NRR200479.
16. Roberfroid M., Gobson G. R., Hoyles L., McCartney A. L., Rastall R., Rowland I., Wolvers D., Watzl B., Szajewska H., Stahl B., et al. Prebiotic effects: Metabolic and health benefits. Br. J. Nutr. 2011; 104:S1-S63.
17. Leach J. D., Sobolik K. D. High dietaryintake of prebiotic inulin-type fructans in the prehistoric Chihauhuan desert. Br. J. Nutr. 2010; 103:1158-1561.
18. Tak W. Mak, Mary E. Saunders, in The Immune Response, 2006.
19. Swain S, McKinstry K K, Strutt™. Expanding roles for CD4⁻ T cells in immunity to viruses. Nat Rev Immunol 2012; 12(2): 136-48.
20. Tay M Z, Poh C M, Renia L, MacAry P A, Ng L F P. The trinity of COVID-19: immunity, inflammation and intervention. Nat Rev Immunol 2020; 20(6): 363-74.
21. Huang C, Wang Y, Li X, et al. Clinical features of patients infected with 2019 novel coronavirus in Wuhan, China. Lancet 2020; 395(10223): 497-506. [Erratum in Lancet 2020; 395(10223): 496.]
22. Peng Y, Mentzer A J, Liu G, et al. Broad and strong memory CD4+ and CD8+ T cells induced by SARS-CoV-2 in U K convalescent individuals following COVID-19 [published online ahead of print, 2020 Sep. 4]. Nat Immunol 2020; 10.1038/s41590-020-0782-6.
23. Sattler A, Angermair S, Stockmann H, et al. SARS-CoV-2 specific T-cell responses and correlations with COVID-19 patient predisposition. J Clin Invest 2020; 140965.
24. Mathew D, Giles J R, Baxter A E, et al. Deep immune profiling of COVID-19 patients reveals patient heterogeneity and distinct immunotypes with implications for therapeutic interventions. Preprint. bioRxiv 2020; 2020.05.20.106401. Published 2020 May 23.
25. Chen Z, John Wherry E. T cell responses in patients with COVID-19. Nat Rev Immunol 2020; 20(9): 529-36.
26. Matthew A. Lunning, Steven M. Horwitz, in Abeloffs Clinical Oncology (Sixth Edition), 2020.
27. Uddin M N, Horvat D, Glaser S, Mitchell B M, Puschett J B (2008). “Examination of the cellular mechanism by which marinobufagenin inhibits cytotrophoblast function.” J. Biol. Chem. 283: 17946-17953.
28. Uddin M N, Childs Ed W, Horvat D, Puschett J B (2009) “Marinobufagenin enhances endothelial permeability via activation of apoptotic signaling.” Am J Physiol Regul Integr Comp Physiol 296: R1726-R1734.
29. Uddin M N, D. Horvat, S. Demorrow, E. Agunanne, J. Puschett (2010). “Marinobufagenin is an upstream modulator of Gadd45a stress signaling in preeclampsia.” Biochim Biophys Acta. 2010 Sep. 17. [Epub ahead of print]
30. Uddin M N, Agunanne E, Horvat D, Puschett J B (2010) “Resibufogenin Administration Prevents Oxidative Stress in a Rat Model of Human Preeclampsia” Hypertens Pregnancy 2010 Dec. 21. [Epub ahead of print].
31. Uddin M N, S. Allen, R. Jones, S. S. Glaser, D. C. Zawieja and T. J. Kuehl (2012) Pathogenesis of preeclampsia: translational aspects: marinobufagenin and angiogenic imbalance as biomarkers of the syndrome. Transl Res. 2012 August; 160(2):99-113. Epub 2012 Feb. 2. An invited featured article review article.
32. Jessica R Ehrig, Horvat D, Leonard D, Allen S R, Jones R O, Kuehl T J, Uddin M N. Cardiotonic steroids induce anti-angiogenic and anti-proliferative profiles in first trimester cytotrophoblast cells. Placenta. 2014 Aug. 7. pii: S0143-4004(14)00641-9. doi: 10.1016/j.placenta.2014.07.014. [Epub ahead of print].
33. Chase R Cawyer, Horvat D, Leonard D, Allen S R, Jones R O, Zawieja D C, Kuehl T J, Uddin M N. Hyperglycemia impairs cytotrophoblast function via stress signaling. Am J Obstet Gynecol. 2014 November; 211(5):541.el-8. doi: 10.1016/j.ajog.2014.04.033. Epub 2014 May 1.
34. Jessica C. Ehrig, S. H. Afroze, M. Reyes Steven R. Allen, Nathan Drever, Kimberly A. Pilkinton, Thomas J. kuehl, Uddin, M N. Cardiotonic steroids induce apoptotic and stress signaling proteins in first trimester cytotrophoblast cells. Placenta. 2015 Sep. 3. pii: S0143-4004(15)30043-6. doi: 10.1016/j.placenta.2015.08.016. [Epub ahead of print]
35. Owen, Judith; Punt, Jenni; Stranford, Sharon (2013). Kuby Immunology. New York: W. H. Freeman and Company. p. 40.
36. Golubovskaya V, Wu L (2016). “Different Subsets of T Cells, Memory, Effector Functions, and CAR-T Immunotherapy”. Cancers. 8 (3): e36. doi:10.3390/cancers8030036. PMC 4810120. PMID 26999211.
37. Huang W, Berube J, McNamara M, Saksena S, O'Gorman M (2020). “Lymphocyte Subset Counts in COVID-19 Patients: A Meta-Analysis”. Cytometry Part A. 97 (8): 772-776. doi:10.1002/cyto.a.24172. PMC 7323417. PMID 32542842.
38. Morrow J D, Frei B, Longmire A W, et al. Increase in circulating products of lipid peroxidation (F2-isoprostane) in smokers. N Engl J Med 1995; 332:1198-1203.
39. Uddin M N; Agunanne E E; Horvat D; Puschett J B, Hypertension in pregnancy [Hypertens Pregnancy], ISSN: 1525-6065, 2012, pp. 70-8; Publisher: Informa Healthcare; PMID: 21174582, Database: MEDLINE Complete PubMed.
40. Ki Hyung Kim, Hye Young Kim, Hyeong Hoe Kim, Kyu Sup Lee, and JaeHun Cheong. Hypoxia induces expression of COX-2 through the homeodomain transcription factor CDX1 and orphan nuclear receptor SHP in human endometrial cells. Molecular Human Reproduction, Vol. 17, No. 11 pp. 710-719, 2011.
41. Kang, H K, Ecklund, D, Liu, M, et al. (2009) Apigenin, a non-mutagenic dietary flavonoid, suppresses lupus by inhibiting autoantigen presentation for expansion of autoreactive Th1 and Th17 cells. Arthritis Res Ther 11, R59.
42. Kim, H P, Son, K H, Chang, H W, et al. (2004) Anti-inflammatory plant flavonoids and cellular action mechanisms. Pharm Sci 96, 229-245.
43. Moreira, A, Fraga, C, Alonso, M, et al. (2004) Quercetin prevents oxidative stress and NF-kappaB activation in gastric mucosa of portal hypertensive rats. Biochem Pharmacol 68, 1939-1946.
44. Gray P W, Goeddel D V (August 1982). “Structure of the human immune interferon gene”. Nature. 298 (5877): 859-63. Bibcode:1982Natur.298.859G. doi:10.1038/298859a0. PMID 6180322. S2CID 4275528.
45. Wheelock E F (July 1965). “Interferon-Like Virus-Inhibitor Induced in Human Leukocytes by Phytohemagglutinin”. Science. 149 (3681): 310-1. Bibcode:1965Sci.149.310W. doi:10.1126/science.149.3681.310. PMID 17838106. S2CID 1366348.
46. “Entrez Gene: INFG”.
47. Schoenborn J R, Wilson C B (2007). “Regulation of Interferon-(During Innate and Adaptive Immune Responses”. Regulation of interferon-gamma during innate and adaptive immune responses. Advances in Immunology. 96. pp. 41-101. doi:10.1016/S0065-2776(07)96002-2. ISBN 978-O-12-373709-0. PMID 17981204.
48. Artis D, Spits H (January 2015). “The biology of innate lymphoid cells”. Nature. 517 (7534): 293-301. Bibcode:2015Natur.517.293A. doi:10.1038/naturel4189. PMID 25592534. S2CID 4386692.
49. Ferguson-Smith A C, Chen Y F, Newman M S, May L T, Sehgal P B, Ruddle F H (April 1988). “Regional localization of the interferon-beta 2/B-cell stimulatory factor 2/hepatocyte stimulating factor gene to human chromosome 7pl5-p21”. Genomics. 2 (3): 203-8. doi:10.1016/0888-7543(88)90003-1. PMID 3294161.
50. “Raised troponin and interleukin-6 levels are associated with a poor prognosis in COVID-19”. Cardiac Rhythm News. 2 Apr. 2020.
51. Meng, Xiao-ming; Nikolic-Paterson, David J.; Lan, Hui Yao (25 Apr. 2016). “TGF-β: the master regulator of fibrosis”. Nature Reviews Nephrology. 12 (6): 325-338. doi:10.1038/nrneph.2016.48. ISSN 1759-5061. PMID 27108839. S2CID 25871413.
52. Massagué J (October 2012). “TGF3 signalling in context”. Nature Reviews. Molecular Cell Biology. 13 (10): 616-30. doi:10.1038/nrm3434. PMC 4027049. PMID 22992590.
53. Nakao A, Afrakhte M, Morén A, Nakayama T, Christian J L, Heuchel R, et al. (October 1997). “Identification of Smad7, a TGFbeta-inducible antagonist of TGF-beta signalling”. Nature. 389 (6651): 631-5. Bibcode:1997Natur.389.631N. doi:10.1038/39369. PMID 9335507. S2CID 4311145.
54. AfCS signaling gateway—data center—ligand description
55. Letterio J J, Roberts A B (April 1998). “Regulation of immune responses by TGF-beta”. Annual Review of Immunology. 16 (1): 137-61. doi:10.1146/annurev.immunol.16.1.137. PMID 9597127.
56. Massagué J, Xi Q (July 2012). “TGF-β control of stem cell differentiation genes”. FEBS Letters. 586 (14): 1953-8. doi:10.1016/j.febslet.2012.03.023. PMC 3466472. PMID 22710171.
57. Li M O, Flavell R A (August 2008). “TGF-beta: a master of all T cell trades”. Cell. 134 (3): 392-404. doi:10.1016/j.cell.2008.07.025. PMC 3677783. PMID 18692464.
58. Massagué J, Blain S W, Lo R S (October 2000). “TGFbeta signaling in growth control, cancer, and heritable disorders”. Cell. 103 (2): 295-309. doi:10.1016/S0092-8674(00)00121-5. PMID 11057902. S2CID 15482063.
59. Kulkarni A B, Huh C G, Becker D, Geiser A, Lyght M, Flanders K C, et al. (January 1993). “Transforming growth factor beta 1 null mutation in mice causes excessive inflammatory response and early death”. Proceedings of the National Academy of Sciences of the United States of America. 90 (2): 770-4.
60. Iszewski M B, Groot A J, Dastych J, Knol E F (May 2007). “TNF trafficking to human mast cell granules: mature chain-dependent endocytosis”. Journal of Immunology. 178 (9): 5701-9. doi:10.4049/jimmunol.178.9.5701. PMID 17442953.
61. Gahring L C, Carlson N G, Kulmar R A, Rogers S W (September 1996). “Neuronal expression of tumor necrosis factor alpha in the murine brain”. Neuroimmunomodulation. 3 (5): 289-303. doi:10.1159/000097283. PMID 9218250.
62. Walsh L J, Trinchieri G, Waldorf H A, Whitaker D, Murphy G F (May 1991). “Human dermal mast cells contain and release tumor necrosis factor alpha, which induces endothelial leukocyte adhesion molecule 1”. Proceedings of the National Academy of Sciences of the United States of America. 88 (10): 4220-4. Bibcode: 1991PNAS.88.4220W. doi:10.1073/pnas.88.10.4220. PMC 51630. PMID 1709737.
63. Feng P, Jyotaki M, Kim A, Chai J, Simon N, Zhou M, Bachmanov A A, Huang L, Wang H (October 2015). “Regulation of bitter taste responses by tumor necrosis factor”. Brain, Behavior, and Immunity. 49: 32-42. doi:10.1016/j.bbi.2015.04.001. PMC 4567432. PMID 25911043.
64. Said E A, Dupuy F P, Trautmann L, Zhang Y, Shi Y, El-Far M, Hill B J, Noto A, Ancuta P, Peretz Y, Fonseca S G, Van Grevenynghe J, Boulassel M R, Bruneau J, Shoukry N H, Routy J P, Douek D C, Haddad E K, Sekaly R P (April 2010). “Programmed death-1-induced interleukin-10 production by monocytes impairs CD4+ T cell activation during HIV infection”. Nat. Med. 16 (4): 452-9. doi:10.1038/nm.2106. PMC 4229134. PMID 20208540.
65. Starkie R, Ostrowski S R, Jauffred S, Febbraio M, Pedersen B K (2003). “Exercise and IL-6 infusion inhibit endotoxin-induced TNF-α production in humans”. FASEB J. 17 (8): 884-886. doi:10.1096/fj.02-0670fje. PMID 12626436. S2CID 30200779.
66. Pedersen B K (December 2009). “The diseasome of physical inactivity—and the role of myokines in muscle-fat cross talk”. J Physiol. 587 (23): 5559-5568. doi:10.1113/jphysiol.2009.179515. PMC 2805368. PMID 19752112.
67. Chadwick W, Magnus T, Mattson M P, Maudsley S (2008). “Targeting TNF-alpha receptors for neurotherapeutics”. Trends in Neurosciences. 31 (10): 504-511. doi:10.1016/j.tins.2008.07.005. PMC 2574933. PMID 18774186.
68. Heir R, Stellwagen D (2020). “TNF-Mediated Homeostatic Synaptic Plasticity: From in vitro to in vivo Models”. Frontiers in Cellular Neuroscience. 14: 565841. doi:10.3389/fncel.2020.565841. PMC 7556297. PMID 33192311.
69. Luster A D, Unkeless J C, Ravetch J V (1985). “Gamma-interferon transcriptionally regulates an early-response gene containing homology to platelet proteins”. Nature. 315 (6021): 672-6. doi:10.1038/315672a0. PMID 3925348. S2CID 4358066.
70. Luster A D, Jhanwar S C, Chaganti R S, Kersey J H, Ravetch J V (May 1987). “Interferon-inducible gene maps to a chromosomal band associated with a (4;11) translocation in acute leukemia cells”. Proceedings of the National Academy of Sciences of the United States of America. 84 (9): 2868-71. doi:10.1073/pnas.84.9.2868. PMC 304761. PMID 2437586.
71. Dufour J H, Dziejman M, Liu M T, Leung J H, Lane T E, Luster A D (April 2002). “IFN-gamma-inducible protein 10 (IP-10; CXCL10)-deficient mice reveal a role for IP-10 in effector T cell generation and trafficking”. Journal of Immunology. 168 (7): 3195-204. doi:10.4049/jimmunol.168.7.3195. PMID 11907072.
72. Angiolillo A L, Sgadari C, Taub D D, Liao F, Farber J M, Maheshwari S, et al. (July 1995). “Human interferon-inducible protein 10 is a potent inhibitor of angiogenesis in vivo”. The Journal of Experimental Medicine. 182 (1): 155-62. doi:10.1084/jem.182.1.155. PMC 2192108. PMID 7540647.
73. Booth V, Keizer D W, Kamphuis M B, Clark-Lewis I, Sykes B D (August 2002). “The CXCR3 binding chemokine IP-10/CXCL10: structure and receptor interactions”. Biochemistry. 41 (33): 10418-25. doi:10.1021/bi026020q. PMID 12173928.
74. Javier González-Gallego, M. Victoria Garcia-Mediavilla, Sonia Sánchez-Campos and Maria J. Tuñón. Fruit polyphenols, immunity and inflammation. British Journal of Nutrition (2010), 104, S15-S27.
75. Raso, G M, Meli, R, Di Carlo, G, et al. (2001) Inhibition of inducible nitric oxide synthase and cyclooxygenase-2 expression by flavonoids in macrophage J774A.1. Life Sci 68, 921-931.
76. Hou, D X, Luo, D, Tanigawa, S, et al. (2007) Prodelphinidin B-4 3′-O-gallate, a tea polyphenol, is involved in the inhibition of COX-2 and iNOS via the downregulation of TAK1-NF-kappaB pathway. Biochem Pharmacol 74, 742-751.
77. Mutoh, M, Takahashi, M, Fukuda, K, et al. (2000) Suppression of cyclooxygenase-2 promoter-dependent transcriptional activity in colon cancer cells by chemopreventive agents with a resorcin-type structure. Carcinogenesis 21, 959-963.
78. Kang, H K, Ecklund, D, Liu, M, et al. (2009) Apigenin, a non-mutagenic dietary flavonoid, suppresses lupus by inhibiting autoantigen presentation for expansion of autoreactive Th1 and Th17 cells. Arthritis Res Ther 11, R59.
79. Kim, H P, Son, K H, Chang, H W, et al. (2004) Anti-inflammatory plant flavonoids and cellular action mechanisms. Pharm Sci 96, 229-245.
80. Moreira, A, Fraga, C, Alonso, M, et al. (2004) Quercetin prevents oxidative stress and NF-kappaB activation in gastric mucosa of portal hypertensive rats. Biochem Pharmacol 68, 1939-1946.
81. Tieppo, J, Cuevas, M J, Vercelino, R, et al. (2009) Prevention of hepatopulmonary syndrome by quercetin administration in cirrhotic rats. J Nutr 139, 1339-1346.
82. Hirano, T, Higa, S, Arimitsu, J, et al. (2004) Flavonoids such as luteolin, fisetin and apigenin are inhibitors of interleukin-4 and interleukin-13 production by activated human basophils. Int Arch Allergy Immunol 134, 135-140.
83. Wadsworth, T L & Koop, D R (1999) Effects of the wine polyphenolics quercetin and resveratrol on pro-inflammatory cytokine expression in RAW 264·7 macrophages. Biochem Pharmacol 57, 941-949.
84. Kim, I B, Kim, D Y, Lee, S J, et al. (2006) Inhibition of IL-8 production by green tea polyphenols in human nasal fibroblasts and A549 epithelial cells. Biol Pharm Bull 29, 1120-1125.
85. Park, H H, Lee, S, Son, H Y, et al. (2008) Flavonoids inhibit histamine release and expression of proinflammatory cytokines in mast cells. Arch Pharm Res 31, 1303-1311.
86. Kim, Y J, Choi, S E, Lee, M W, et al. (2008) Taxifolin glycoside inhibits dendritic cell responses stimulated by lipopolysaccharide and lipoteichoic acid. J Pharm Pharmacol 60, 1465-1472.
87. Sternberg, Z, Chadka, K, Lieberman, A, et al. (2008) Quercetin and interferon-beta modulate immune response(s) in peripheral blood mononuclear cell isolated from multiple sclerosis patients. J Neuroimmunol 205, 142-147.
88. Li, C Y, Suen, J L, Chiang, B L, et al. (2006) Morin promotes the production of Th2 cytokine by modulating bone marrow-derived dendritic cells. Am J Chin Med 34, 667-684.
89. Lee, J S, Kim, S G, Kim, K H, et al. (2007) Silibilin polarizes Th1/Th2 immune responses through the inhibition of immunostimulatory function of dendritic cells. J Cell Physiol 210, 385-397.
90. Comalada, M, Ballester, I, Bailón, E, et al. (2006) Inhibition of pro-inflammatory markers in primary bone marrow-derived mouse macrophages by naturally occurring flavonoids: analysis of the structure-activity relationship. Biochem Pharmacol 72, 1010-1021.
91. Crouvezier, S, Powell, B, Keir, D, et al. (2005) The effects of phenolic components of tea on the production of pro- and anti-inflammatory cytokines by human leukocytes in vitro. Cytokine 13, 280-286.
92. Wang, J, Zhang, Q, Jin, S, et al. (2008) Genistein modulate immune responses in collage-induced reumathoid arthiritis model. Maturitas 59, 405-412.
93. Yano, S, Umeda, D, Yamashita, T, et al. (2007) Dietary flavones suppress IgE and Th2 cytokines in OVA-immunized BALB/c mice. Eur J Nutr 46, 257-263.
94. Park, H J, Lee, C M, Jung, I D, et al. (2009) Quercetin regulates Th1/Th2 balance in a murine model of asthma. Int Immunopharmacol 9, 261-267.
95. Yu, C S, Lai, K C, Yang, J S, et al. (2010) Quercetin inhibited murine leukemia WEHI-3 cells in vivo and promoted immune response. Phytother Res 24, 163-168.
96. Lin, J P, Yang, J S, Lu, C C, et al. (2009) Rutin inhibits the proliferation of murine leukemia WEHI-3 cells in vivo and promotes immune responses in vivo. Leuk Res 33, 823-828.
97. Min, K, Yoon, W K, Kim, S K, et al. (2007) Immunosuppressive effects of silibilin in experimental autoimmune encephalomyelitis. Arch Pharm Res 30, 1265-1272.
98. Ramiro-Puig, & Castell, M (2009) Cocoa: antioxidant and immunomodulator. Br J Nutr 101, 931-940.
99. Kowalski, J, Samojedny, A, Paul, M, et al. (2006) Apigenin inhibit release and gene expression of monocyte chemoattractant protein 1 (MCP-1) in J774·2 macrophages. Wiad Lek 59, 634-638.
100. Ahn, H Y, Xu, Y & Davidge, S T (2008) Epigallocatechin-3-O-gallate inhibits TNFα-induced monocyte chemotactic protein-1 production from vascular endothelial cells. Life Sci 82, 964-968.
101. Ruiz, P A, Braune, A, Holzlwimmer, G, et al. (2007) Quercetin inhibits TNF-induced NF-kappaB transcription factor recruitment to proinflammatory gene promoters in murine intestinal epithelial cells. J Nutr 137, 1208-1215.
102. Erlejman, A G, Jaggers, G, Fraga, C G, et al. (2008) TNFα-induced NF-κB activation and cell oxidant production are modulated by hexameric procyanidins in Caco-2 cells. Arch Biochem Biophys 476, 186-195.
103. Sivaramakrishnan, V & Niranjali Devaraj, S (2009) Morin regulates the expression of NF-κB-p65, COX-2 and matrix metalloproteinases in diethylnitrosamine induced rat hepatocellular carcinoma. Chem Biol Interact 180, 353-359.
104. Choi, S Y, Hwang, J H, Ko, H C, et al. (2007) Nobiletin from citrus fruit peel inhibits the DNA-binding activity of NF-κB and ROS production in LPS-activated RAW264·7 cells. J Ethnopharmacol 113, 149-155.
105. Crespo, I, Garcia-Mediavilla, M V, Gutiérrez, B, et al. (2008) A comparison of the effects of kaempferol and quercetin on cytokine-induced pro-inflammatory status of cultured human endothelial cells. Br J Nutr 100, 968-976.
106. Xu, L, Zhang, L, Bertucci, A M, et al. (2008) Apigenin a dietary flavonoid sensitizes human T cells for activation-induced cell death by inhibiting PKB/Akt and NF-κB activation pathway. Immunol Lett 121, 74-83.
107. Rangan, G K, Wang, Y & Harris, D C (2002) Dietary quercetin augments activator protein-1 and does not reduce nuclear factor-kappa B in the renal cortex of rats with established chronic glomerular disease. Nephron 90, 313-319.CrossRefGoogle Scholar
108. Jang, S, Kelley, K W & Johnson, R W (2008) Luteolin reduces IL-6 production in microglia by inhibiting JNK phosphorylation and activation of AP-1. Proc Natl Acad Sci USA 105, 7534-7539.
109. Hamlinen, M, Nieminen, R, Vuorela, P, et al. (2007) Anti-inflammatory effects of flavonoids: genistein, kaempferol, quercetin, and daidzein inhibit STAT-1 and NF-κB activations, whereas flavones, rhamnetin, naringenin, and pelargonidin inhibit only NF-κB activation along with the inhibitory effects on iNOS expression and NO production in activated macrophages. Mediators Inflamm 2007, 45673-45683.
110. Cai, F, Li, C R, Wu, J L, et al. (2006) Theaflavin ameliorates cerebral ischemia-reperfusion injury in rats through its anti-inflammatory effect and modulation of STAT-1. Mediators Inflamm 2006, 30490.
111. Rezai-Zadeh, K, Ehrhart, J, Bai, Y, et al. (2008) Apigenin and luteolin modulate microglial activation via inhibition of STAT1-induced CD40 expression. J Neuroinflamm 25, 41.
112. Ma, L, Gao, H Q, Li, B Y, et al. (2007) Grape seed proanthocyanidin extracts inhibit vascular cell adhesion molecule expression induced by advanced glycation end products through activation of peroxisome proliferators-activated receptor gamma. J Cardiovasc Pharmacol 49, 293-298.
113. Wadsworth, T L & Koop, D R (2001) Effects of Ginkgo biloba extract (EGb 761) and quercetin on lipopolysaccharide-induced release of nitric oxide. Chem Biol Interact 137, 43-58.
114. Ying, B, Yang, T, Song, X, et al. (2009) Quercetin inhibits IL-1 beta-induced ICAM-1 expression in pulmonary epithelial cell line A549 through the MAPK pathways. Mol Biol Rep 36, 1825-1832.
115. Terao, J. Factors modulating bioavailability of quercetin-related flavonoids and the consequences of their vascular function. Biochem. Pharmacol. 2017, 139, 15-23, DOI: 10.1016/j.bcp.2017.03.021
116. Henning, S. M.; Wang, P.; Abgaryan, N.; Vicinanza, R.; de Oliveira, D. M.; Zhang, Y.; Lee, R.; Carpenter, C. L.; Aronson, W. J.; Heber, D. Phenolic acid concentrations in plasma and urine from men consuming green or black tea and potential chemopreventive properties for colon cancer. Mol. Nutr. Food Res. 2013, 57, 483-493, DOI: 10.1002/mnfr.201200646
117. Hamming, I.; Timens, W.; Bulthuis, M.; Lely, A.; Navis, G.; van Goor, H. Tissue distribution of ACE2 protein, the functional receptor for SARS coronavirus. A first step in understanding SARS pathogenesis. J. Pathol. 2004, 203, 631-637, DOI: 10.1002/path. 1570
118. de Boer, V. C. J.; Dihal, A. A.; van der Woude, H.; Arts, I. C. W.; Wolffram, S.; Alink, G. M.; Rietjens, I. M. C. M.; Keijer, J.; Hollman, P. C. H. Tissue distribution of quercetin in rats and pigs. J. Nutr. 2005, 135, 1718-1725, DOI: 10.1093/jn/135.7.1718
119. Mullen, W.; Rouanet, J.-M.; Auger, C.; Teissedre, P.-L.; Caldwell, S. T.; Hartley, R. C.; Lean, M. E. J.; Edwards, C. A.; Crozier, A. Bioavailability of [2-14C]quercetin-4′-glucoside in rats. J. Agric. Food Chem. 2008, 56, 12127-12137, DOI: 10.1021/jf802754s 120. Ivey, K. L.; Hodgson, J. M.; Croft, K. D.; Lewis, J. R.; Prince, R. L. Flavonoid intake and all-cause mortality. Am J. Clin. Nutr. 2015, 101, 1012-1020, DOI: 10.3945/ajcn.113.073106
121. Serban, M. C.; Sahebkar, A.; Zanchetti, A.; Mikhailidis, D. P.; Howard, G.; Antal, D.; Andrica, F.; Ahmed, A.; Aronow, W. S.; Muntner, P. Effects of quercetin on blood pressure: a systematic review and meta-analysis of randomized controlled trials. J. Am. Heart Assoc. 2016, 5, e002713 DOI: 10.1161/JAHA.115.002713
122. Tamtaji, O. R.; Milajerdi, A.; Dadgostar, E.; Kolahdooz, F.; Chamani, M.; Amirani, E.; Mirzaei, H.; Asemi, Z. The effects of quercetin supplementation on blood pressures and endothelial function among patients with metabolic syndrome and related disorders: A systematic review and meta-analysis of randomized controlled trials. Curr. Pharm. Des. 2019, 25, 1372-1384, DOI: 10.2174/1381612825666190513095352
123. Bondonno, N. P.; Bondonno, C. P.; Hodgson, J. M.; Ward, N. C.; Croft, K. D. The efficacy of quercetin in cardiovascular health. Curr. Nutr. Rep. 2015, 4, 290-303, DOI: 10.1007/s13668-015-0137-3
124. Patel, R. V.; Mistry, B. M.; Shinde, S. K.; Syed, R.; Singh, V.; Shin, H.-S. Therapeutic potential of quercetin as a cardiovascular agent. Eur. J. Med. Chem. 2018, 155, 889-904, DOI: 10.1016/j.ejmech.2018.06.053
125. Larson, A. J.; Symons, J. D.; Jalili, T. Quercetin: A treatment for hypertension?-A review of efficacy and mechanisms. Pharmaceuticals 2010, 3, 237-250, DOI: 10.3390/ph3010237
126. Islam, M.; Schmidt, R.; Gunaseelan, S.; Sanchez, A. An update on the cardiovascular effects of quercetin, a plant flavonoid. Curr. Nutr. Food Sci. 2014, 10, 36-48, DOI: 10.2174/157340131001140328115216
127. Actis-Goretta, L.; Ottaviani, J. I.; Fraga, C. G. Inhibition of angiotensin converting enzyme activity by flavanol-rich foods. J. Agric. Food Chem. 2006, 54, 229-234, DOI: 10.1021/jf052263o
128. Hussain, F.; Jahan, N.; Rahman, K.-u.; Sultana, B.; Jamil, S. Identification of hypotensive biofunctional compounds of Coriandrum sativum and evaluation of their angiotensin-converting enzyme (ACE) inhibition potential. Oxid. Med. Cell. Longevity 2018, 2018, 4643736 DOI: 10.1155/2018/4643736
129. Hettihewa, S. K.; Hemar, Y.; Rupasinghe, H. Flavonoid-rich extract of actinidia macrosperma (a wild kiwifruit) inhibits angiotensin-converting enzyme in vitro. Foods 2018, 7, 146, DOI: 10.3390/foods7090146
130. Häckl, L. P. N.; Cuttle, G.; Dovichi, S. S.; Lima-Landman, M.; Nicolau, M. Inhibition of angiotensin-converting enzyme by quercetin alters the vascular response to bradykinin and angiotensin L Pharmacology 2002, 65, 182-186, DOI: 10.1159/000064341
131. Neto-Neves, E. M.; Montenegro, M. F.; Dias-Junior, C. A.; Spiller, F.; Kanashiro, A.; Tanus-Santos, J. E. Chronic treatment with quercetin does not inhibit angiotensin-converting enzyme in vivo or in vitro. Basic Clin. Pharmacol. Toxicol. 2010, 107, 825-829, DOI: 10.1111/j.1742-7843.2010.00583.x
132. McAnulty, S. R.; McAnulty, L. S.; Morrow, J. D.; Khardouni, D.; Shooter, L.; Monk, J.; Gross, S.; Brown, V. Effect of daily fruit ingestion on angiotensin converting enzyme activity, blood pressure, and oxidative stress in chronic smokers. Free Radical Res. 2005, 39, 1241-1248, DOI: 10.1080/10715760500306836
133. Larson, A.; Witman, M. A.; Guo, Y.; Ives, S.; Richardson, R. S.; Bruno, R. S.; Jalili, T.; Symons, J. D. Acute, quercetin-induced reductions in blood pressure in hypertensive individuals are not secondary to lower plasma angiotensin-converting enzyme activity or endothelin-1: nitric oxide. Nutr. Res. 2012, 32, 557-564, DOI: 10.1016/j.nutres.2012.06.018
134. Kuba, K.; Imai, Y.; Rao, S.; Gao, H.; Guo, F.; Guan, B.; Huan, Y.; Yang, P.; Zhang, Y.; Deng, W. A crucial role of angiotensin converting enzyme 2 (ACE2) in SARS coronavirus-induced lung injury. Nat. Med. 2005, 11, 875-879, DOI: 10.1038/nm1267
135. Li, G.; He, X.; Zhang, L.; Ran, Q.; Wang, J.; Xiong, A.; Wu, D.; Chen, F.; Sun, J.; Chang, C. Assessing ACE2 expression patterns in lung tissues in the pathogenesis of COVID-19. J. Autoimmun. 2020, 102463 DOI: 10.1016/j.jaut.2020.102463

### Example 2: Influence of the Absorption of Superoxide Dismutase by Modulating Gut Microbes

### Background

Superoxide dismutase (SOD) is a primary antioxidant enzyme with a very high molecular weight which normally has issues with physical stability, stomach acid degradation and, most importantly, absorption. Even though SOD is beneficial to reduce cellular oxidative stress and can be helpful to many aging related dysfunctions, it has been difficult to produce an orally effective SOD dosage form. REVIVIFY® contains a diverse group of functional molecules which influence each other's bioavailability including SOD absorption. The acid resistant stability and absorption of SOD is influenced by microbial modulation, which is provided by certain components of the REVIVIFY® gel—prebiotic fiber and polyphenols. In order to prove this hypothesis, a study was performed to evaluate whether SOD alone and SOD as par of the REVIVIFY® composition cause any microbial modulation. The study below shows that changes in microbial composition of the gut occur, which cause SOD absorption and effectiveness.

### Experimental Methods

The first objective of this work was to develop a scalable in vitro model for the maintenance of gut microbiome profiles. Bacterial cells are cultured in 96-deep well plates and covered with a silicone-gel cover perforated at the top of each well. This cover facilitates gas-exchange with the outer environment in the chamber, so as to preserve the partial pressure of gases and volatile metabolites in each well, which could subsequently preserve certain levels of dissolved gas molecules in the culture medium.

Treatment Conditions: 1. SOD alone; 2. Fibersol (digestion-resistant maltodextrin) alone; 3. Polyphenols alone; 4. SOD+Fibersol; 5. SOD+polyphenols; and 6. REVIVFY finished product. Using these treatments it is possible to determine how Fibersol and Polyphenols influence the absorption of SOD using an in vitro guy microbe system.

Stool Specimen Collection and Processing: Briefly, approx. 3 g of fresh stool sample was collected from each individual using a 2.5 ml sterile sampling spoon (Bel-Art, United States). Each spoon is dropped into a 50 ml Falcon tube containing 15 ml of sterile PBS pre-reduced with 0.1% (w/v) L-cysteine hydrochloride. The samples were immediately transferred into an anaerobic workstation (5% H₂, 5% CO₂, and 90% N₂ at 37° C.). Microbiomes were characterized at 0 (immediately after inoculation), 3, 6, 9, 12, 24, 34, and 48 h by measurements of the optical density at 595 nm (OD595) as a proxy of microbial growth and biomass, and by metaproteomic analyses. Results were determined using both goat gut and silicone mat microbiome systems.

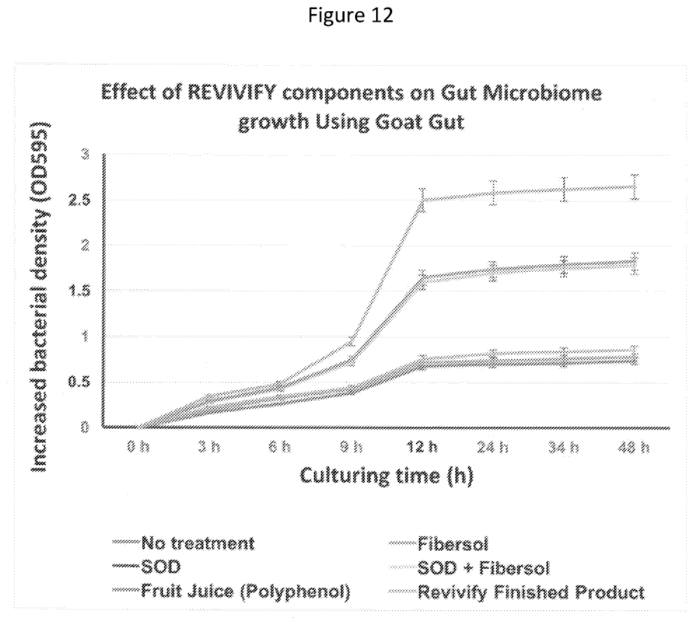

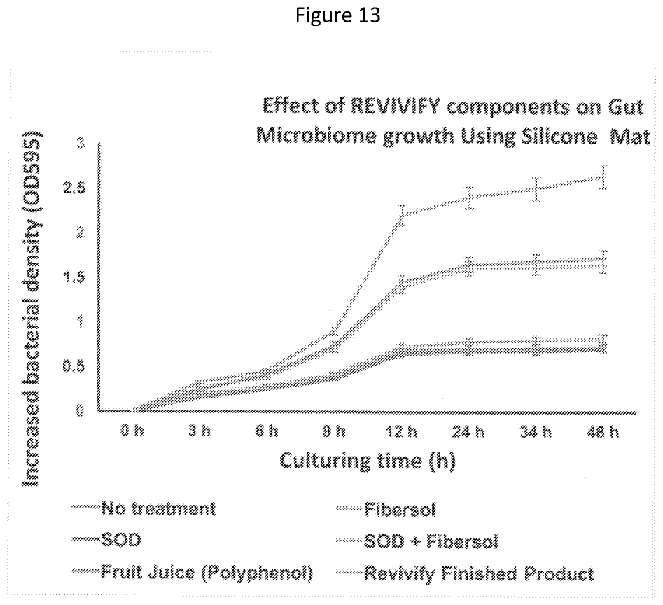

Results: The bacterial growth gradually increases with time. The Fibersol and SOD alone sample do not influence bacterial growth compared to control as shown by OD measurements (FIG. 12, Goat Gut System; FIG. 13, Silicone Mat System). In both FIGS. 12 and 13, the top line in the plot is for the REVIVIFY® finished product, the middle two lines are for the fruit juice polyphenols only and the SOD+Fibersol and the bottom three lines are for the no treatment control, SOD only and Fibersol only. However, the polyphenols in fruit juice significantly increased bacterial growth with the REVIVIFY® finished product causing the greatest increase in OD. These experiments demonstrated that Fibersol augmented the absorption of SOD via both silicone mat and fresh goat guy experiments as demonstrated by the increased bacterial growth.

Conclusion: This study shows the development of a scalable in vitro model for the maintenance of gut microbiome profiles. Moreover, the absorption patterns of different components of REVIVFY gel are shown, including the influence of Fibersol on the absorption of SOD. This in vitro model of gut microbes can be used for evaluation of other drugs, prebiotics or nutraceuticals.

### Example 3: Modulation of Healthy Gut Microbiomes and Short Chain Fatty Acids Evaluated by an In Vitro Gut Microbiome Model

Background: Gut Health is very important for healthy living and well-being. The microbial community in the gut plays major role in the immune system, hormonal processes, neurological conditions, metabolism, mineral absorption, vitamin production and several cellular processes. According to the American Diabetes Association, gut microbiota is known to effect host physiology within and outside the gut. Gut microbiota is essential for homeostasis of immune system in the gut, modulation of epithelial proliferation, and protection against opportunistic bacteria. The gut microbes present within gastrointestinal tract have coevolved within the human host to perform a number of functions the host would otherwise be unable to accomplish on its own The major gut microbes are of phylum Firmicutes and Bacteroidetes, followed by Actinobacteria and Protobacteria. The beneficial effects of gut-microbes can be measured by the production of Short chain fatty acids (SCFAs), mainly acetate, propionate, and butyrate, and lactic acid. This production depends on the type of fiber consumption. In this instance the fiber is a soluble fiber known as Fibersol-2 along with mixed fructo-oligosaccharide from various fruit concentrates. The hypothesis is that there should be modulation of beneficiary microbes with an increased ratio of short chain fatty acids that protect host health in multiple ways. This study evaluates the effect of REVIVIFY® finished product on gut microbiome modulation and short chain fatty acids by an in vitro model of gut microbiome study.

Experimental Method: An in vitro Gut Microbiome Culture Model was established as described in Example 2 above. The first objective of this work is to develop a scalable in vitro model for the maintenance of gut microbiome profiles. In vitro models that maintain the functional and compositional profiles of in vivo gut microbiomes would be extremely valuable. In vitro model experiments were performed as described in Example 2. Gut microbes were cultured in 2 ml 96-well plates and treated with control, SOD, Prebiotic fiber, Fruit juice, or the Finished REVIVIFY® product for 24 hours. Cultured microbiome samples were harvested at 24 h for metaproteomic analysis. Afterwards, a culture aliquot was collected for chemical analysis (SCFA content) and microbiome profiling.

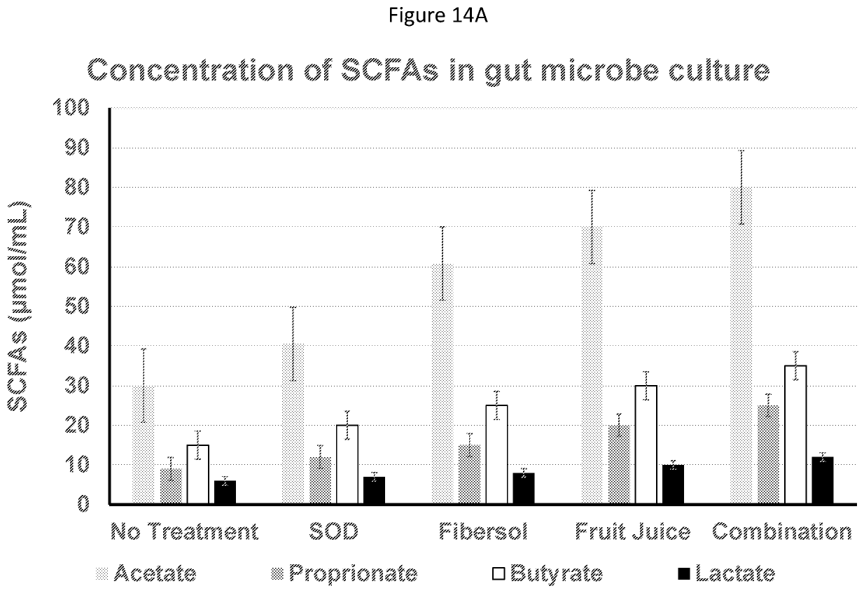

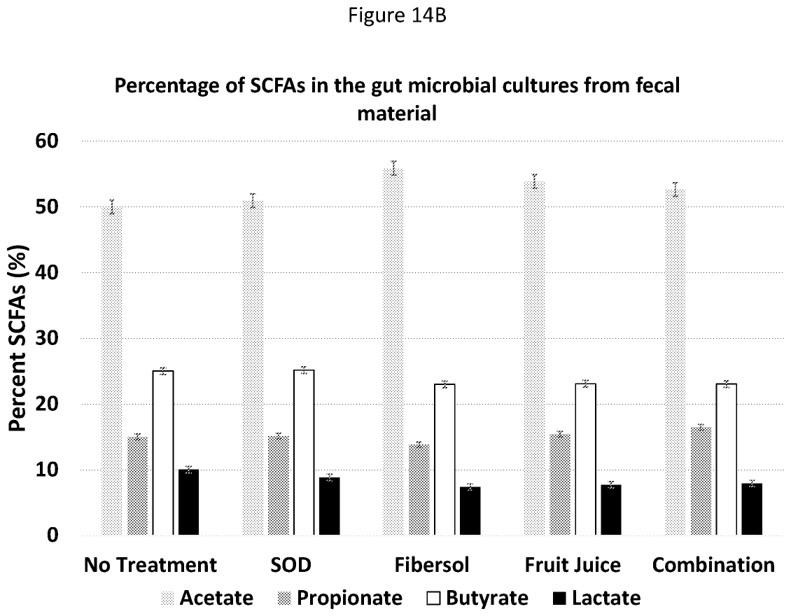

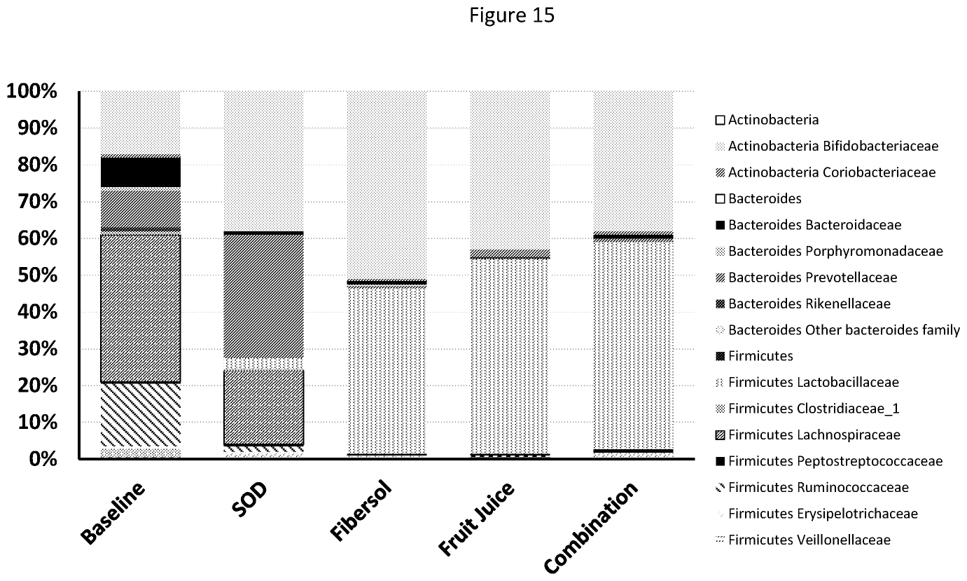

Results: This study evaluated the change in gut microbial composition and the SCFAs when they were treated with four different compounds (SOD, Prefibrotic fiber, Fruit juice, and Finished product). As seen in FIG. 14, results show that the amount of SCFAs significantly increases when treated with the finished product (FIG. 14A) but the ratio of the SCFAs remain the same across all the treatment groups including the control (FIG. 14B). In the control treatment, the concentration of acetate, propionate, butyrate, and lactate is 30 μmol/ml, 9 μmol/ml, 15 μmol/ml, and 6 μmol/ml respectively which increases to 80 μmol/ml, 25 μmol/ml, 35 μmol/ml, and 12 μmol/ml when treated with the finished product for 24 hours. This is an unexpectedly large 2.5-fold increase in SCFAs in response to the finished product. When calculating the ratio of these SCFAs across control and all four-treatment groups, it appeared that the acetate, propionate, butyrate, and lactate ratios remained consistently around 53%, 15%, 24%, and 8% respectively (See FIG. 14). Our study also demonstrated that Prefibrotic fiber, Fruit juice, and Finished product promoted the growth of two good gut microbes, Firmicutes lactobacillus and Actinobacteria Bifidobacteriaceae when compared to the baseline and SOD (See FIG. 15; where Firmicutes lactobacillus is represented by vertical hatching and Actinobacteria Bifidobacteriaceae is represented by light grey shading). Both Lactobacillus and Bifidobacterium are good microbes and are present in many food preparation like yogurt for digestibility and immunity. Out of these two the shifting trend is towards lactobacillus which reflects the production of lactic acid in SCFAs graph. Also, Lactobacillus belongs to the Firmicutes spp. Normally these are butyrate producers, which is reflected in the SCFA results. The microbiome profile of the culture treated with the finished product showed significant increase of lactobacillus in Firmicutes spp. followed by the Bifidobacterium in Actinobacterium species in the gut microbial community.

Conclusions/Perspectives: Dietary prebiotics are selectively fermented ingredient that results in specific changes in the compositions of two beneficial microbiota of lactobacillus in Firmicutes species and Bifidobacterium in Actinobacteria species. This is significant finding where both the beneficial microbes can contribute the host a positive influences by exerting homofermentative and heterofermentative outcomes along with digest and metabolize protein and carbohydrate, synthesis of B-vitamins as well as vitamin K, catabolism of bile salts, enhance innate innate as well as acquired immunity, inhibit pro-inflammatory mediators, anti-bacterial activities against array of pathogens such as Pseudonomas, Candida, E. Coli, Aurous, Salmonella, also Shigella, C. difficile, and Helicobactor pylori. Lactobacillus can be bio-marker of vaginal health, they are the major part of vaginal microbiota. This study demonstrates that the REVIVIFY® finished product elevated the amount of SCFAs in the gut whereas keeping the ratio of each SCFA (acetate, propionate, butyrate, and lactate) consistent with the control. This result exhibits a balanced increase of the SCFAs without in a consistent manner that improves and maintains a healthy colon environment. The study suggests that REVIVIFY® is a unique dietary supplement which produces relatively higher in butyrate contributing many health benefits intestinal epithelium cells integrity, immune cells integrity and response, enteric neurons bi-directional signaling of gut-brain axis, and nutrients production and metabolism. Butyrate is major energy source for colonocytes and is involved in the maintenance of colonic mucosal health.

### Example 4: Attenuation of Oxidative Damage of Human Brain Microvascular Endothelial Cells (HBMEC)

### Introduction

Accumulating data suggests that oxidative stress and mitochondrial damage are involved in the pathogenesis of neurodegenerative disorders including Parkinson Disease (PD), Multiple Sclerosis (MS), Alzheimer's Disease (AD), and many others. The brain uses about 20% of oxygen consumption, and is thus a high producer of reactive oxygen species (ROS). Also, the brain cell membrane is composed of more unsaturated fatty acids (MUFA and PUFA), and thus more prone to lipid auto-oxidation due to ROS. REVIVIFY® gel can provide instant reduction of oxidative stress from multi-dimensional pathways and an immediate effect induced by the disease symptoms.

The REVIVIFY® formulation neutralizes major oxidants of superoxide anion, hydroxyl radicals, singlet oxygen, peroxy-nitrite, peroxy-radicals, and hypochlorite.

### Background

Superoxide dismutases (SODs): SODs constitute a very important antioxidant defense against oxidative stress in the body. The enzyme acts as a good therapeutic agent against reactive oxygen species-mediated diseases. The present review describes the therapeutic effects of SOD in various physiological and pathological conditions such as cancer, inflammatory diseases, cystic fibrosis, ischemia, aging, rheumatoid arthritis, neurodegenerative diseases, and diabetes. However, the enzyme has certain limitations in clinical applications. Therefore, SOD conjugates and mimetics have been developed to increase its therapeutic efficiency^{1,2,3,4,5,6,7}.

Polyphenols: Polyphenols are secondary metabolites of plants and are generally involved in defense against ultraviolet radiation or aggression by pathogens. In the last decade, there has been much interest in the potential health benefits of dietary plant polyphenols as antioxidant. Epidemiological studies and associated meta-analyses strongly suggest that long term consumption of diets rich in plant polyphenols offer protection against development of cancers, cardiovascular diseases, diabetes, osteoporosis and neurodegenerative diseases^{8,9,10,11,12}.

Dietary prebiotic fibers: The health benefits of dietary fiber have long been appreciated. Higher intakes of dietary fiber are linked to less cardiovascular disease and fiber plays a role in gut health, with many effective laxatives actually isolated fiber sources. Higher intakes of fiber are linked to lower body weights. Only polysaccharides were included in dietary fiber originally, but more recent definitions have included oligosaccharides as dietary fiber, not based on their chemical measurement as dietary fiber by the accepted total dietary fiber (TDF) method, but on their physiological effects. Inulin, fructo-oligosaccharides, and other oligosaccharides are included as fiber in food labels in the US. Additionally, oligosaccharides are the best known “prebiotics”, “a selectively fermented ingredient that allows specific changes, both in the composition and/or activity in the gastrointestinal microflora that confers benefits upon host well-bring and health.” To date, all known and suspected prebiotics are carbohydrate compounds, primarily oligosaccharides, known to resist digestion in the human small intestine and reach the colon where they are fermented by the gut microflora. Studies have provided evidence that inulin and oligofructose (OF), lactulose, and resistant starch (RS) meet all aspects of the definition, including the stimulation of Bifidobacterium, a beneficial bacterial genus. Other isolated carbohydrates and carbohydrate-containing foods, including galactooligosaccharides (GOS), transgalactooligosaccharides (TOS), polydextrose, wheat dextrin, acacia gum, psyllium, banana, whole grain wheat, and whole grain corn also have prebiotic effects^{13,14,15,16,17}.

The purpose of this study is to evaluate whether REVIVIFY® gel attenuates human brain microvascular endothelial cells (HBMEC) from oxidative damage. The following biomarkers were evaluated in a hypoxia-induced HBMEC culture media:
1. Malondialdehyde (MDA)—a biomarker of lipid oxidative damage
2. 4-Hydroxynonenal, or 4-hydroxy-2-nonenal or 4-HNE or HNE—a biomarker of lipid peroxidation
3. Protein Carbonyls—a biomarker of protein oxidation
4. 3-nitrotyrosine—a biomarker of cell damage linked to degeneration of dopamine neurons

Malondialdehyde (MDA) is an organic compound of the formula CH₂(CHO)₂. A colorless liquid, malondialdehyde is a highly reactive compound that occurs as the enol.[1] It occurs naturally and is a marker for oxidative stress.

4-Hydroxynonenal, or 4-hydroxy-2-nonenal or 4-HNE or HNE, (C₉H₁₆O₂), is an α,β-unsaturated hydroxyalkenal that is produced by lipid peroxidation in cells. 4-HNE is the primary alpha,beta-unsaturated hydroxyalkenal formed in this process. 4-HNE has 3 reactive groups: an aldehyde, a double-bond at carbon 2, and a hydroxy group at carbon 4.

Protein Carbonyls: Protein carbonyl (PC) content in blood and tissues are a reliable indicator of protein oxidation. Traumatic brain injury (TBI) results from an impact to the head that disrupts normal brain function. Severe TBI can cause permanent brain damage or death. Diffuse axonal injury (DAI) is a typical pathological change after TBI and is closely associated with clinical prognosis. DAI has two distinct pathological features: swellings and large terminal bulbs due to excessive neurofilament aggregation. Secondary axonal injury resulting from cytoskeleton abnormalities is the most common cause of DAI.

Oxidative stress is a well-known factor implicated in DAI, and the mitochondrial phosphorylating capacity, concentrations of the nicotinic coenzyme pool, and oxidative/nitrosative stress correlate closely with the severity of DAI. Carbonyl modification occurs as a direct result of oxidative damage to proteins, leading to protein dysfunction and the formation of protein aggregates. Protein carbonylation has been shown to contribute to the pathogenesis of several neurodegenerative diseases such as multiple sclerosis, Parkinson's disease, and Alzheimer's disease. Under normal conditions, carbonylated proteins are thought to be degraded by proteasomes whose main function is to recognize and degrade unneeded, damaged, or misfolded proteins. Under prooxidative conditions, however, the increased generation of reactive oxygen species (ROS) or reactive carbonyl species may reduce the activity of proteasomes, leading to the accumulation of carbonylated proteins in affected cells.

3-nitrotyrosine: Nitrotyrosine is a product of tyrosine nitration mediated by reactive nitrogen species such as peroxynitrite anion and nitrogen dioxide. Nitrotyrosine is identified as an indicator or marker of cell damage, inflammation as well as NO (nitric oxide) production. Nitrotyrosine is formed in the presence of the active metabolite NO. Generally in many disease states, oxidative stress increases the production of superoxide (O₂⁻) and NO forming peroxynitrite (ONOO⁻) a destructive free radical oxidant. The production of ONOO⁻ is capable of oxidizing several lipoproteins and of nitrating tyrosine residues in many proteins. It is difficult to determine the production of ONOO⁻ so, usually nitrotyrosine in proteins are the detectable marker for indirectly detecting ONOO⁻. It is detected in large number of pathological conditions and is considered a marker of NO-dependent, reactive nitrogen species-induced nitrative stress. Nitrotyrosine is detected in biological fluids such as plasma, lung aspirants-BALF (Broncho alveolar lining fluid) and urine. Increased level of nitrotyrosine is detected in rheumatoid arthritis septic shock and coeliac disease. In all these studies nitrotyrosine was undetected in healthy subjects. Nitrotyrosine is also found in numerous other disease-affected tissues, such as the cornea in keratoconus. Peroxynitrite and/or nitrative stress may participate in the pathogenesis of diabetes.

Nitrotyrosine, as a marker of reactive oxygen species, has also been linked to degeneration of dopamine neurons. Tyrosine is the precursor to dopamine, a neurotransmitter that's important for motivation, attention, learning, circadian rhythms, and other biological processes.

Study Design: Human brain microvascular endothelial cells (HBMEC) were cultured in a six well plate with low oxygen condition (hypoxia condition; 2% oxygen). Control HBMEC cells were cultured under normal oxygen concentration conditions. Prior to treatment, cells were incubated in serum free media for 24 hours. Cells were treated for 48 hours with following agents: 1. Superoxide Dismutase only; 2. Prebiotic fiber only; 3. Fruit juice only; 4. superoxide Dismutase+Prebiotic fiber+Fruit juice (Combination); and 5. Negative Control.

Enzyme-Linked Immunosorbent Assay: After the 48 h incubation, the media was removed from cells and placed in tubes. To evaluate whether revivify gel attenuates human brain microvascular endothelial cells (HBMEC) from oxidative damage, the following biomarkers were evaluated in a hypoxia-induced HBMEC culture media: 1. Malondialdehyde (MDA); 2. 4-Hydroxynonenal, or 4-hydroxy-2-nonenal or 4-HNE or HNE; 3. Protein Carbonyls; and 4. 3-nitrotyrosine.

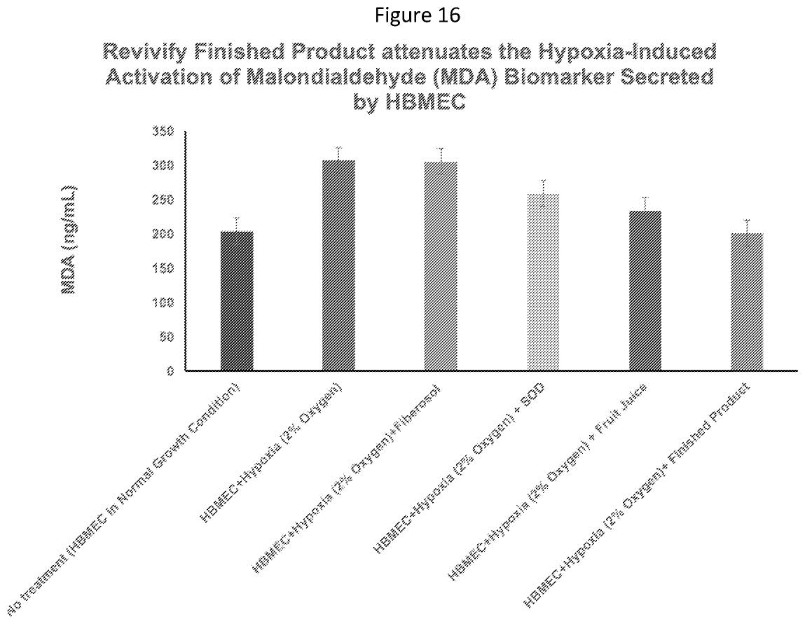

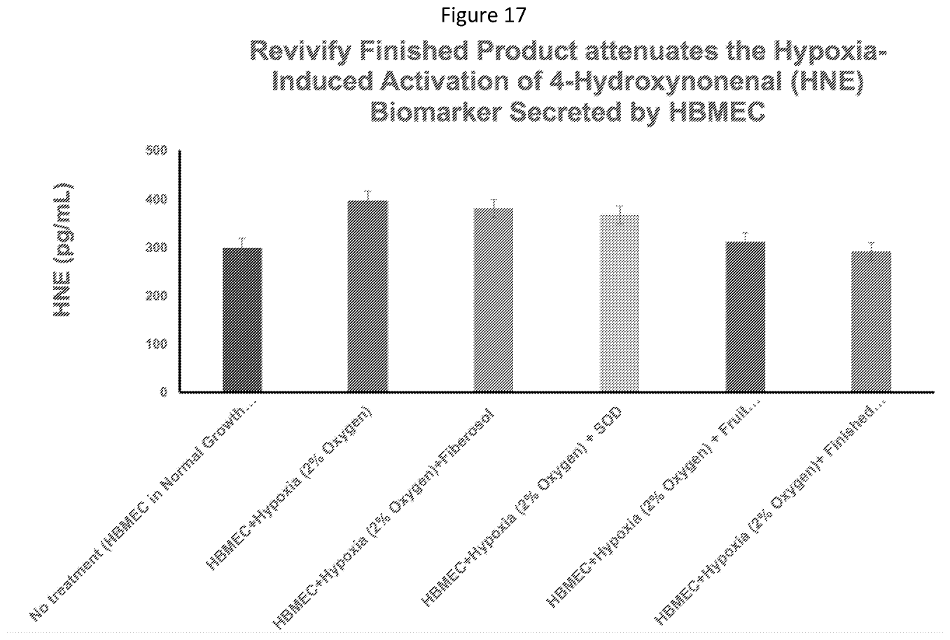

Results: As is seen in FIGS. 16-19, while some of the components of the gel showed improvements in oxidative damage biomarkers, the greatest improvements were seen with the finished product gel. As seen in FIG. 16, each component of the gel provides some reduction of the MDA biomarker compared to positive control, with the finished product gel surprisingly having MDA levels under hypoxic conditions that are lower than the cells cultured under non-hypoxic control conditions. Similar results are seen in FIG. 17 with HNE as the biomarker, where, once again, the finished product gel surprisingly has lower biomarker levels than the cells cultured under non-hypoxic control conditions.

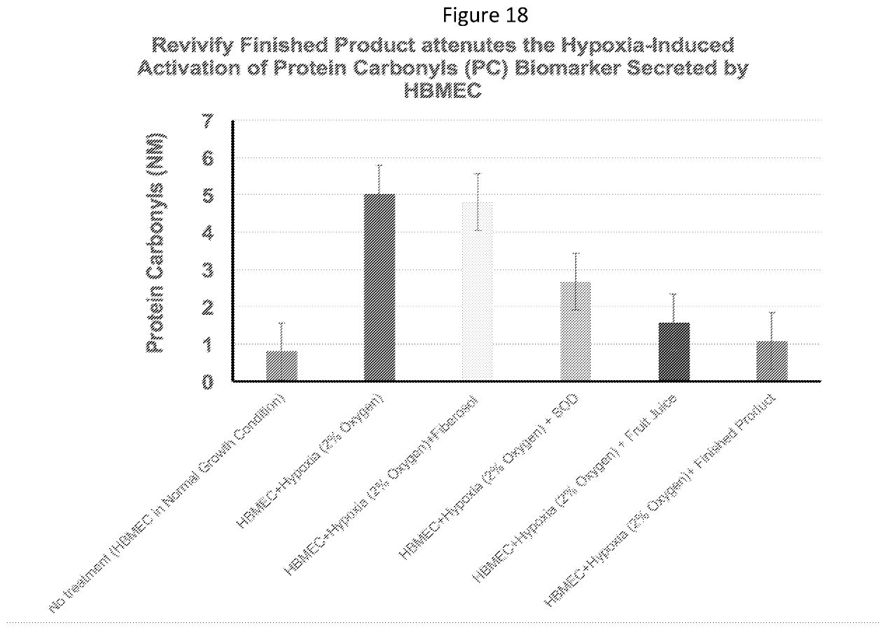

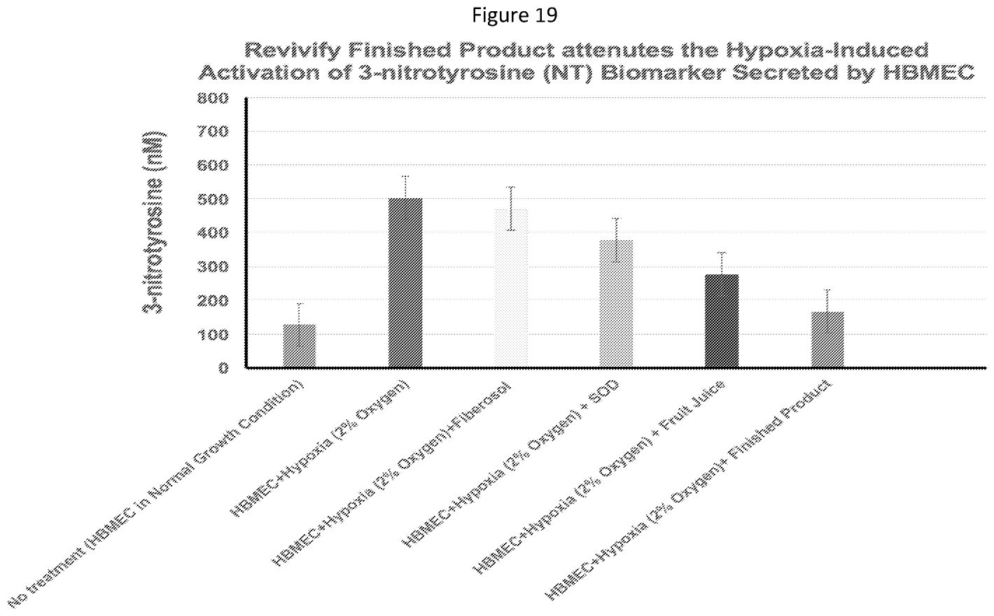

As seen in FIG. 18, levels of protein carbonyls biomarkers are reduced from positive control for each gel component individually, with a surprising and significant reduction from positive control for the finished gel product. Similar results are observed for the 3-nitrotyrosine biomarker as shown in FIG. 19, with the finished product again showing surprising and significant reduction compared to positive control.

The results in this Example demonstrate that the finished REVIVIFY@gel provides substantial and surprising protection of HBMECs from oxidative agents. These results suggest that absorbed SOD, when combined with a soluble fiber and polyphenols, can provide significant protection of nervous system cells from oxidative damage.

### REFERENCES FOR EXAMPLE 4

1. Kangralkar V A, Patil S D, Bandivadekar R M. Oxidative stress and diabetes: A review. Intl J Pharm Appl. 2010; 1:38-45.
2. Yasui K, Baba A. Therapeutic potential of superoxide dismutase (SOD) for resolution of inflammation. Inflamm Res. 2006; 55:359-63.
3. Landis G N, Tower J. Superoxide dismutase evolution and life span regulation. Mech Ageing Dev. 2005; 126:365-79.
4. Noor R, Mittal S, Iqbal J. Superoxide dismutase—Applications and relevance to human diseases. Med Sci Monit. 2002; 8:RA210-5.
5. Inal M E, Kanbak G, Sunal E. Antioxidant enzyme activities and malondialdehyde levels related to aging. Clin Chim Acta. 2001; 305:75-80.
6. Riley D P. Functional mimics of superoxide dismutase enzymes as therapeutic agents. Chem Rev. 1999; 99:2573-88.
7. Salvemini D, Riley D P. Nonpeptidyl mimetics of superoxide dismutase in clinical therapies for diseases. Cell Mol Life Sci. 2000; 57:1489-92.
8. Scalbert A, Manach C, Morand C, Remesy C. Dietary polyphenols and the prevention of diseases. Crit Rev Food Sci Nutr. 2005; 45:287-306.
9. Beckman C H. Phenolic-storing cells: keys to programmed cell death and periderm formation in wilt disease resistance and in general defense responses in plants?Physiol. Mol. Plant Pathol. 2000; 57:101-110.
10. Arts I C W, Hollman P C H. Polyphenols and disease risk in epidemiologic studies. Am J Clin Nutr. 2005; 81:317-325.
11. Kondratyuk T P, Pezzuto J M. Natural Product Polyphenols of Relevance to Human Health. Pharm Biol. 2004; 42:46-63.
12. Shahidi F, Naczk M. Food phenolics, sources, chemistry, effects, applications. Lancaster, P A: Technomic Publishing Co Inc; 1995.
13. Slavin J. L. Dietary fiber: Classification, chemical analyses, and food sources. J. Am. Diet. Assoc. 1987; 87:1164-1171.
14. Gibson G. R., Roberfroid M. B. Dietary modulation of the human colonic microbiota: Introducing the concept of prebiotics. J. Nutr. 1995; 125:1401-1412.
15. Gibson G. R., Probert H. M., van Loo J., Rastall R. A., Roberfroid M. B. Dietary modulation of the human colonic microbiota: Updating the concept of prebiotics. Nutr. Res. Rev. 2004; 17:259-275. doi: 10.1079/NRR200479.
16. Roberfroid M., Gobson G. R., Hoyles L., McCartney A L., Rastall R., Rowland I., Wolvers D., Watzl B., Szajewska H., Stahl B., et al. Prebiotic effects: Metabolic and health benefits. Br. J. Nutr. 2011; 104:S1-S63.
17. Leach J. D., Sobolik K. D. High dietaryintake of prebiotic inulin-type fructans in the prehistoric Chihauhuan desert. Br. J. Nutr. 2010; 103:1158-1561.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A liquid composition comprising:
a) from about 0.03 units/mL to about 0.5 units/mL superoxide dismutase;
b) from about 1.3 mg/mL to about 23 mg/mL soluble fiber; and
c) water.

2. The liquid composition of claim 1, comprising from about 0.05 units/mL to about 0.4 units/mL superoxide dismutase.

3. The liquid composition of claim 1, comprising from about 0.2 units/mL to about 0.3 units/mL superoxide dismutase.

4. The liquid composition of any one of claims 1-3, comprising from about 2.7 mg/mL to about 12 mg/mL soluble fiber.

5. The liquid composition of any one of claims 1-3, comprising from about 5.55 mg/mL to about 11.11 mg/mL soluble fiber.

6. The liquid composition of any one of claims 1-5, wherein the superoxide dismutase is extracted from melon, bovine liver, heterotrophic bacteria or marine phytoplankton.

7. The liquid composition of any one of claims 1-6, wherein the superoxide dismutase is a copper/zinc superoxide dismutase, an iron/manganese superoxide dismutase or a nickel superoxide dismutase.

8. The liquid composition of any one of claims 1-7, wherein the ratio of superoxide dismutase to soluble fiber is from about 1:100 to about 1:1000 by weight.

9. The liquid composition of any one of claims 1-7, wherein the ratio of superoxide dismutase to soluble fiber is from about 1:500 to about 1:700 by weight.

10. The liquid composition of any one of claims 1-9, wherein the soluble fiber is a water soluble polysaccharide.

11. The liquid composition of any one of claims 1-10, wherein the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides.

12. The liquid composition of any one of claims 1-10, wherein the soluble fiber is soluble corn fiber.

13. The liquid composition of claim 12, wherein the soluble corn fiber is digestion-resistant maltodextrin.

14. The liquid composition of any one of claims 1-13, further comprising from about 0.1 mg/mL to about 1.5 mg/mL of a simple sugar.

15. The liquid composition of any one of claims 1-13, further comprising from about 0.1 mg/mL to about 1.5 mg/mL d-ribose.

16. The liquid composition of any one of claims 1-13, further comprising from about 0.40 mg/mL to about 0.85 mg/mL d-ribose.

17. The liquid composition of any one of claims 1-16, further comprising from about 1.3 mg/mL to about 9.0 mg/mL of a sugar alcohol.

18. The liquid composition of any one of claims 1-16, further comprising from about 1.3 mg/mL to about 9.0 mg/mL erythritol.

19. The liquid composition of any one of claims 1-16, further comprising from about 2.7 mg/mL to about 5.6 mg/mL erythritol.

20. The liquid composition of any one of claims 1-19, further comprising from about 0.1 mg/mL to about 1.5 mg/mL of a pH adjusting agent.

21. The liquid composition of any one of claims 1-19, further comprising from about 0.1 mg/mL to about 1.5 mg/mL citric acid.

22. The liquid composition of any one of claims 1-19, further comprising from about 0.4 mg/mL to about 0.7 mg/mL citric acid.

23. The liquid composition of any one of claims 1-22, further comprising from about 0.05 mg/mL to about 0.75 mg/mL of a sweetener.

24. The liquid composition of any one of claims 1-22, further comprising from about 0.05 mg/mL to about 0.75 mg/mL steviol glycoside.

25. The liquid composition of any one of claims 1-22, further comprising from about 0.2 mg/mL to about 0.35 mg/mL steviol glycoside.

26. The liquid composition of any one of claims 1-22, further comprising a flavoring.

27. A composition comprising:
a) from about 10 units to about 200 units superoxide dismutase;
b) from about 500 mg to about 8000 mg soluble fiber; and
c) a probiotic.

28. The composition of claim 27, comprising from about 50 units to about 150 units superoxide dismutase.

29. The composition of claim 27, comprising from about 70 units to about 100 units superoxide dismutase.

30. The composition of any one of claims 27-29, comprising from about 1000 mg to about 5000 mg soluble fiber.

31. The composition of any one of claims 27-29, comprising from about 2000 mg to about 4000 mg soluble fiber.

32. The composition of any one of claims 27-31, wherein the superoxide dismutase is extracted from melon, bovine liver, heterotrophic bacteria or marine phytoplankton.

33. The composition of any one of claims 27-31 wherein the superoxide dismutase is a copper/zinc superoxide dismutase, an iron/manganese superoxide dismutase or a nickel superoxide dismutase.

34. The composition of any one of claims 27-33, wherein the ratio of superoxide dismutase to soluble fiber is from about 1:100 to about 1:1000 by weight.

35. The composition of any one of claims 27-33, wherein the ratio of superoxide dismutase to soluble fiber is from about 1:500 to about 1:700 by weight.

36. The composition of any one of claims 27-35, wherein the soluble fiber is a water soluble polysaccharide.

37. The composition of any one of claims 27-35, wherein the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides.

38. The composition of any one of claims 27-35, wherein the soluble fiber is soluble corn fiber.

39. The composition of claim 38, wherein the soluble corn fiber is digestion-resistant maltodextrin.

40. The composition of any one of claims 27-39, wherein the probiotic comprises bacteria of the genus Bifidobacterium.

41. The composition of any one of claims 27-39, wherein the probiotic comprises bacteria of the genus Lactobacillus.

42. The composition of any one of claims 27-39, wherein the probiotic comprises Firmicutes lactobacillus, Actinobacteria Bifdobacteriaceae or combinations thereof.

43. The composition of any one of claims 27-39, wherein the composition is in the form of a gel.

44. The composition of any one of claims 27-39, wherein the composition is in the form of a liquid.

45. The composition of any one of claims 27-39, wherein the composition is in the form of a powder.

46. A method of increasing T-cell activation in a subject comprising orally administering to the subject a composition comprising:
a) from about 10 units to about 200 units superoxide dismutase; and
b) from about 500 mg to about 8000 mg soluble fiber;
wherein, following administration of the composition, the activation of T cells is increased in the subject.

47. The method of claim 46, wherein the composition is administered in combination with an anti-cancer agent.

48. The method of claim 46, wherein the composition is administered in combination with an anti-viral agent.

49. A method of increasing the production of short chain fatty acids (SCFAs) in the digestive tract of a subject comprising orally administering to the subject a composition comprising:
a) from about 10 units to about 200 units superoxide dismutase; and
b) from about 500 mg to about 8000 mg soluble fiber;
wherein, following administration of the composition, the production of SCFAs is increased in the digestive tract of the subject.

50. The method of claim 49, wherein the SCFAs increased in production are acetate, propionate, butyrate, or lactate SCFAs, or combinations thereof.

51. The method of claim 50, wherein SCFAs are increased in a manner that provides about the same ratio of acetate, propionate, butyrate, and lactate SCFAs compared to the ratio of acetate, propionate, butyrate, and lactate SCFAs prior to the increase.

52. A method of increasing the amount of bacteria of the genus Bifidobacterium or Lactobacillus in the digestive tract of a subject comprising orally administering to the subject a composition comprising:
a) from about 10 units to about 200 units superoxide dismutase; and
b) from about 500 mg to about 8000 mg soluble fiber;
wherein, following administration of the composition, the amount of bacteria of the genus Bifidobacterium, Lactobacillus, or combinations thereof, is increased in the digestive tract of the subject.

53. The method of claim 52, wherein the bacteria of the genus Bifidobacterium comprise the species Actinobacteria Bifidobacteriaceae.

54. The method of claim 52, wherein the bacteria of the genus Lactobacillus comprise the species Firmicutes lactobacillus.

55. The method of any one of claims 46-54, wherein the composition comprises from about 50 units to about 150 units superoxide dismutase.

56. The method of any one of claims 46-54, wherein the composition comprises from about 70 units to about 100 units superoxide dismutase.

57. The method of any one of claims 46-56, wherein the composition comprises from about 1000 mg to about 5000 mg soluble fiber.

58. The method of any one of claims 46-56, wherein the composition comprises from about 2000 mg to about 4000 mg soluble fiber.

59. The method of any one of claims 46-58, wherein the superoxide dismutase is extracted from melon, bovine liver, heterotrophic bacteria or marine phytoplankton.

60. The method of any one of claims 46-58, wherein the superoxide dismutase is a copper/zinc superoxide dismutase, an iron/manganese superoxide dismutase or a nickel superoxide dismutase.

61. The method of any one of claims 46-60, wherein the ratio of superoxide dismutase to soluble fiber in the composition is from about 1:100 to about 1:1000 by weight.

62. The method of any one of claims 46-60, wherein the ratio of superoxide dismutase to soluble fiber in the composition is from about 1:500 to about 1:700 by weight.

63. The method of any one of claims 46-62, wherein the soluble fiber is a water soluble polysaccharide.

64. The method of any one of claims 46-62, wherein the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides.

65. The method of any one of claims 46-62, wherein the soluble fiber is soluble corn fiber.

66. The method of claim 65, wherein the soluble corn fiber is digestion-resistant maltodextrin.

67. The method of any one of claims 46-66, wherein the composition is in the form of a gel.

68. The method of any one of claims 46-66, wherein the composition is in the form of a liquid.

69. The method of any one of claims 46-66, wherein the composition is in the form of a powder.
